(19) United States
(12) Patent Application Publication
(10) Pub. No.: US 2016/0201045 A1
(43) Pub. Date: Jul. 14, 2016

(54) Endo-xylanase and Coding Gene and Use Thereof

(71) Applicant: SHANGHAI INSTITUTES FOR BIOLOGICAL SCIENCES CHINESE ACADEMY OF SCIENCES, Shanghai (CN)

(72) Inventors: Zhihua ZHOU, Shanghai (CN); Qianfu WANG, Shanghai (CN); Wei WEI, Shanghai (CN); Changli QIAN, Shanghai (CN); Qian WANG, Shanghai (CN); Ning LIU, Shanghai (CN); Lei XIE, Shanghai (CN); Xing YAN, Shanghai (CN); Yongping HUANG, Shanghai (CN)

(21) Appl. No.: 14/911,911

(22) PCT Filed: Aug. 14, 2014
(86) PCT No.: PCT/CN2014/084349

§ 371 Date: Feb. 12, 2016

(30) Foreign Application Priority Data
Aug. 15, 2013 (CN) 201310357541.5

(51) Int. Cl. C12N 9/24

(52) U.S. Cl. CPC C12N 9/2482; C12Y 302/01008

(57) ABSTRACT

Provided are an endo-xylanase and a coding gene and the use thereof. Also provided are an expression vector and a host cell containing the coding gene, a method for forming a simple sugar by using the xylanase, a xylanase mutant with an improved thermal stability and a method for improving the thermal stability of the xylanase.

### TECHNICAL FIELD

The present disclosure belongs to the biotechnical field, relating to a novel endo-xylanase, its coding gene and use thereof.

### TECHNICAL BACKGROUND

Brief Introduction of Xylan. The backbone of xylan is formed by xylose molecules (D xylose) linked through β-1,4-glycosidic linkage and the branched chains are formed by arabinofuranosidic group, glucuronyl or acetyl, etc. Xylan is the major component of the hemicellulose in the plant cell wall. Besides cellulose, hemicellulose is the second important component of the plant polysaccharide and is the second abundant reproducible plant polysaccharide in the nature.

Source of Xylan. Materials rich in xylan could be obtained from various sources, including agricultural, forestry and industrial wastes, such as hardwood, cork, stalk, straw, bran, etc., and municipal solid wastes, etc. Different plants contain different amounts of xylan. Hardwood contains more xylan than cork. The amount of xylan may be accounted for 15˜30% of the dry weight of hardwood and 7˜12% of the dry weight of cork commonly. The amount of xylan may be up to 30% or more in some annual plants, such as wheat, sugarcane, and cotton seed hull.

Brief Introduction of Xylanase. Xylanase is a collective term of a series of glycosyl hydrolases that can specifically degrade xylan. Many enzymes are required to thoroughly degrade xylan due to differences in monosaccharide units that consist of the xylan, in types of bonds, and in branched chains of xylan having different substituents. The enzymes include endo-β-1,4-xylanase (EC 3.2.1.8), β-xylosidase (EC 3.2.1.37), α-L-arabinofuranosidase (E.C. 3.2.1.39), β-D-glucuronidase (EC 3.2.1.39), acetyl xylan esterases (E.C. 3.1.1.72), and ferulic or p-coumaric acid esterase (E.C. 3.2.1.73) that degrades the ester bond formed by residue of the side chain of arabinose and phenolic acid (such as ferulic acid or coumaric acid), etc. Among the above enzymes, the endo-β-1,4-xylanase is the major enzyme for degrading xylan, which acts on the internal β-1,4-xylosidic linkage within the backbone of xylan through an endo-cleavage manner to hydrolyze the large polyxylan to oligoxylan and small-amount xyloses, thereby initiating the gradual degradation of polysaccharide (Bernier R, Driguez H, Desrochers M Gene 26:59-65, 1983).

Application of Xylanase in Traditional Industry. Xylanase is widely used and plays an important role in various industrial sectors, including food, feed, paper manufacture, and spinning, etc. Firstly, in the food industry, xylanase is used in processing of fruit, vegetable and plant to promote the dipping procedure, to make the juice clear and to increase the output and filter efficiency. Xylanase is also used in the preparation and brewage of grape wine to promote dipping of grape skin and to reduce turbidity of the finished product. Xylanase is also used in the baking, grinding, and processing of cookie and candy to increase the elasticity and strength of flour dough and to improve the texture of the bread. It is also used in the coffee processing to reduce the viscosity of the coffee extract and improve the drying/freeze-drying procedure. Secondly, in the industry of paper manufacture, xylanase is used to promote the pulping treatment and to replace the mechanical pulping, which can not only effectively reduce energy consumption but also increase formation of fibril of pulp and water permeability, thereby increasing processing efficiency and paper strength. Thirdly, in the spinning industry, xylanase is used in the enzymolysis of textiles, such as flax, jute, ramie, hemp, etc., to reduce or replace the chemical blending method. Fourthly, in the agriculture and animal husbandry, xylanase is widely used in the feed for monogastric animals, such as pig and poultry, and ruminants, to assist the animal to effectively degrade xylan, reduce the content of the non-starch polysaccharide in the feed, increase the digestibility and nutritive value of the feed, and reduce environmental pollution.

Application of Xylanase in Bioenergy Field. Importantly, xylanase can be used together with other cellulases and hemicellulases in the industrial production of converting lignocellulose to fuel ethanol, under the background that global fossil resources are increasingly depleted and development of new bioenergy is imminent. In one aspect, xylanase could greatly increase frequency and efficiency of cellulase in contacting and acting on cellulose chain by degrading the hemicellulose chain closely crosslinked with lignin and cellulose backbone in lignocellulose, thereby indirectly increasing the degradation efficiency of cellulose. In another aspect, with research and development of pentose fermentation pathway and strains in recently years, the process of producing the fuel ethanol by utilizing bacteria, yeast and filamentous fungi to ferment the hydrolyzed product, xylose, of xylan is mature gradually. With the above two aspects, the conversion efficiency of lignocellulose is greatly improved and thus the cost for producing fuel ethanol is effectively reduced.

Study History of Xylanase. Since xylanase could be widely used, research on xylanase began early at 1960′. A lot of xylanases with different types and functions were isolated from different sources of microbes. Xylanases from Trichoderma reesei, Aspergillus niger, Streptomyces lividans, Cellulomonas fimi, Clostridium thermocellum, and Penicillium simplicissimum, etc., have been clearly investigated. What should be noted is that most of these xylanase genes were isolated from the pure culture of microbes. However, species of microbes in the nature that can be cultured are less than 1% and the resultant xylanases are far from enough to meet the needs of modern industrial production in their physical and chemical properties, catalytic efficiency and yield, etc.

Considering that most of the xylanases known in the prior art exhibit a relatively low activity, and their physical and chemical properties, catalytic efficiency and yield, etc., are far from enough to meet the needs of modern industrial production, there is a need to further broaden the objects to be screened and to screen out novel xylanases having a high enzymatic activity for industrial production to increase the production efficiency.

### SUMMARY

The purpose of the present disclosure is to provide a novel endo-xylanase, its coding gene and use thereof.

The purpose of the present disclosure is to provide an expression vector and a host cell comprising the endo-xylanase gene, methods for expressing the gene and purifying the protein, and the zymological characteristics and functional features of the recombinant protein.

In one aspect of the present disclosure, an isolated polypeptide is provided, which is selected from the group consisting of:

(a) a polypeptide set forth in SEQ ID NO:2;

(b) a polypeptide fragment consisting of amino acid residues 19-272 of SEQ ID NO:2;

(c) a polypeptide fragment consisting of amino acid residues 19-267 of SEQ ID NO:2;

(d) a polypeptide comprising amino acids 19-267 of SEQ ID NO:2;

(e) a polypeptide formed by substitution, deletion or insertion of one or several, such as 1-20, preferably 1-10, more preferably 1-5, and more preferably 1-3, amino acids in the amino acid sequence of (a), (b), (c) or (d) and having the function of the polypeptide of (a);

(f) a polypeptide formed by adding a tag sequence or a signal peptide sequence at the N or C terminus of the polypeptide of (a), (b), (c), (d) or (e);

(g) a fusion protein containing the polypeptide of (a), (b), (c), (d) or (e).

In a preferred embodiment, the polypeptide is derived from an intestinal metagenomics library of Globitermes sulphureus, which is one of the high termite species.

In a specific embodiment, the function of the polypeptide in (a) includes but is not limited to the function of being used as an endo-xylanase.

In a preferred embodiment, the polypeptide of (e) exhibits an improved thermal stability as compared to the wild type sequence, in addition to the function of being used as an endo-xylanase.

In a specific embodiment, the polypeptide is selected from the group consisting of:

(i) SEQ ID NO:2; and

(ii) a fragment of SEQ ID NO:2 which contains at least amino acids 19-267 of SEQ ID NO:2.

In a specific embodiment, the fragment consists of amino acid residues 19-272 of SEQ ID NO:2.

In a specific embodiment, the polypeptide of (e) is selected from the group consisting of polypeptides containing amino acid substitution(s) at the site(s) corresponding to at least one of K32, N37, S42, M80, K205, E219, A221, M222, K223, T228 and A386 of SEQ ID NO:2.

In a specific embodiment, the polypeptide of (e) contains substitutions at least at K32 and K223 of SEQ ID NO:2. In some specific embodiments, the substitution mutation is a combination of K32T with any of K223M, K223E, K223T, K223C, K223S, K223G and K223L.

In a specific embodiment, the polypeptide of (e) contains substitution at least at position 223, which is selected from the group consisting of K223M, K223E, K223T, K223C, K223S, K223G and K223L.

In a specific embodiment, the mutation in the polypeptide of (e) is a substitution selected from one or more of K32T, N37D, S42N, M80I, K205E, E219D, A221T, M222L, K223M, K223E, K223T, K223C, K223S, K223G, K223L, T228S, and A386S.

In a specific embodiment, the polypeptide of (e) is selected from the group consisting of (1) the polypeptide in which a substitution mutation, N37D, is present at position corresponding to amino acid residue 37 of SEQ ID NO:2; (2) the polypeptide in which a substitution mutation, S42N, is present at position corresponding to amino acid residue 42 of SEQ ID NO:2; (3) the polypeptide in which a substitution mutation, M80I, is present at position corresponding to amino acid residue 80 of SEQ ID NO:2; (4) the polypeptide in which a substitution mutation, E219D, is present at position corresponding to amino acid residue 219 of SEQ ID NO:2; (5) the polypeptide in which a substitution mutation, A221T, is present at position corresponding to amino acid residue 221 of SEQ ID NO:2; (6) the polypeptide in which a substitution mutation, M222L, is present at position corresponding to amino acid residue 222 of SEQ ID NO:2; (7) the polypeptide in which a substitution mutation, K223M, is present at position corresponding to amino acid residue 223 of SEQ ID NO:2; (8) the polypeptide in which a substitution mutation, T228S, is present at position corresponding to amino acid residue 228 of SEQ ID NO:2; (9) the polypeptide in which substitution mutations, K205E, K223T and A386S, are present at positions corresponding to amino acid residues 205, 223 and 386 of SEQ ID NO:2; (10) the polypeptide in which substitution mutations, K32T and K223T, are present at positions corresponding to amino acid residues 32 and 223 of SEQ ID NO:2; (11) the polypeptide in which substitution mutations, K205E and K223T, are present at positions corresponding to amino acid residues 205 and 223 of SEQ ID NO:2; (12) the polypeptide in which a substitution mutation, K223E, K223T, K223C, K223S, K223G or K223L, is present at position corresponding to amino acid residue 223 of SEQ ID NO:2; (13) the polypeptide in which substitution mutations, K21T and K223C, are present at positions corresponding to amino acid residues 21 and 223 of SEQ ID NO:2; and (14) the polypeptide in which substitution mutations, K32T and K223S, are present at positions corresponding to amino acid residues 32 and 223 of SEQ ID NO:2.

In another aspect of the present disclosure, an isolated polynucleotide is provided, which contains a nucleotide sequence selected from the group consisting of:

(1) a polynucleotide encoding the polypeptide;

(2) a polynucleotide complementary to the polynucleotide of (1).

In another preferred embodiment, the polynucleotide encodes the polypeptide set for in SEQ ID NO:2.

In another preferred embodiment, the nucleotide sequence of the polynucleotide is set forth in SEQ ID NO:1.

In another aspect of the present disclosure, a vector is provided, which contains the polynucleotide.

In another aspect of the present disclosure, a genetically engineering host cell is provided, which contains the vector, or in which the polynucleotide is integrated into its genome.

In another aspect of the present disclosure, a method for preparing the polypeptide is provided, which comprises:

(a) culturing the host cell;

(b) isolating the polypeptide from the culture.

In another aspect of the present invention, a method is provided for degrading xylan or materials containing xylan to xylo-oligosaccharide or monosaccharide by utilizing the polypeptide.

In another preferred embodiment, the xylo-oligosaccharide is xylobiose, xylotriose or xylotetraose.

In another preferred embodiment, the polypeptide degrades the substrate through an endo-cleavage manner, and the substrate is xylan, or materials containing xylan, such as hemicellulose.

In another preferred embodiment, the xylan is birch xylan or beech xylan.

In another aspect of the present disclosure, a composition is provided, which comprises a safe and effective amount of the polypeptide and a bromatologically acceptable or industrially acceptable carrier.

In another preferred embodiment, the composition further comprises additives for regulating enzymatic activity.

In another preferred embodiment, the additives for regulating enzymatic activity are additives that improve enzymatic activity, preferably selected from the group consisting of K⁺, Mn²⁺, or materials that could be hydrolyzed to form K⁺ or Mn²⁺ after adding to the substrate; or the additives for regulating enzymatic activity are additives that inhibit enzymatic activity, preferably selected from the group consisting of Ni²⁺, Zn²⁺, Fe³⁺ and EDTA, or materials that could be hydrolyzed to form Ni²⁺, Zn²⁺ or Fe³⁺ after adding to the substrate.

In a preferred embodiment, the xylan is birch xylan or beech xylan.

In another preferred embodiment, the substrate to be hydrolyzed is treated by the polypeptide at pH 3-12, such as 3.5-9.5, 5-10, 4-9.5, 5.5-9.5, preferably 6.0-9.5, more preferably 6.0-8.0, most preferably 7.0.

In another preferred embodiment, the substrate to be hydrolyzed is treated by the polypeptide under 15-90□, such as 25-80□, preferably 30-60□, more preferably 45-55□, further more preferably 50-55□.

In another preferred embodiment, additives for regulating enzymatic activity are added during treatment with the polypeptide.

In another preferred embodiment, the additives for regulating enzymatic activity are additives that improve enzymatic activity, preferably selected from the group consisting of K⁺, Mn²⁺, or materials that could be hydrolyzed to form K⁺ or Mn²⁺ after adding to the substrate; or

the additives for regulating enzymatic activity are additives that inhibit enzymatic activity, preferably selected from the group consisting of Ni²⁺, Zn²⁺, Fe³⁺ and EDTA, or materials that could be hydrolyzed to form Ni²⁺, Zn²⁺ or Fe³⁺ after adding to the substrate.

The present disclosure also provides a method for improving the thermal stability of xylanase, comprising mutating the amino acid residue(s) of the xylanase polypeptide at position(s) corresponding to amino acid residue 32 and/or 223 of SEQ ID NO:2, thereby obtaining xylanase having an improved thermal stability.

In a specific embodiment, the xylanase polypeptide is the xylanase polypeptide known in the art.

In other embodiments, the xylanase polypeptide is SEQ ID NO:2 of the present disclosure or active fragment(s) thereof

In other embodiments, the mutation further comprises mutations at other positions of xylanase. In preferred embodiments, the other positions include one or more of positions 37, 42, 80, 205, 219, 221, 222, 228 and 386, numbered according to the amino acid positions in SEQ ID NO:2.

The present disclosure further provides a method for screening a xylanase having an improved thermal stability, comprising:

(1) constructing a library comprising mutants of SEQ ID NO:2 or its active fragments; and

(2) testing the thermal stability of the mutants in the library;

wherein, after testing under the same conditions, the mutant having a reduction degree of activity lower than that of the control by at least 5%, preferably at least 10%, at least 20%, at least 30% or more is the mutant having an improved thermal stability.

In the above method, the control may be the starting polypeptide used for constructing the mutant library, such as SEQ ID NO:2 or its fragment(s) that contain amino acids 19-267 or 19-272, or some mutants ascertained to have the endo-xylanase activity of SEQ ID NO:2 and the same or improved thermal stability as compared to SEQ ID NO:2 or its fragment(s) that contain amino acids 19-267 or 19-272.

In a specific embodiment, the constructed mutants at least contain substitution mutation(s) at one or more positions selected from the group consisting of K32, N37, S42, M80, K205, E219, A221, M222, K223, T228 and A386, numbered according to the amino acid positions in SEQ ID NO:2.

In a specific embodiment, the constructed mutants at least include mutation(s) at K32 and/or K223. In other embodiments, the constructed mutants can further comprise mutation(s), preferably substitution, at one or more positions selected from the group consisting of N37, S42, M80, K205, E219, A221, M222, T228 and A386. The above positions are numbered according to SEQ ID NO:2.

In a specific embodiment, the thermal stability test includes testing the enzymatic activity of the mutants and control at pH 3-12, preferably 5.5-10, more preferably about 7.0, and 15-90° C., preferably 30-60° C., more preferably 50-55° C., and wherein the substrate for testing is selected from birch xylan and beech xylan.

Other aspects of the present disclosure will be apparent to the skilled artisan in view of the contents disclosed in the present disclosure.

### BRIEF DESCRIPTION OF DRAWINGS

FIG. 1 shows the electrophoretogram obtained after PCR of recombinant E. coli BL21(DE3)/pET28a-xyl7 colonies. Lane M shows the electrophoretic result of DNA marker, respectively corresponding to 23.1 kb, 9.4 kb, 6.6 kp, 4.4 kp, 2.3 kb, 2.0 kb, 564 bp bands, from top to bottom. Lanes 1-5 indicate the electrophoretogram of five monoclonal colonies of E. coli BL21(DE3)/pET28a(+)-xyl7 obtained after PCR.

FIG. 2 shows the SDS-PAGE diagram obtained after expression of the endo-1,4-β-xylanase gene xyl7 and purification of the expressed product. Lane 1: proteins from the supernatant of cell lysate; lane 2: 20 mM imidazole eluent; lane 3: 40 mM imidazole eluent; lane 4: 60 mM imidazole eluent; lane 5: 100 mM imidazole eluent; lane 6: 200 mM imidazole eluent; lane 7: 500 mM imidazole eluent; lane M: protein Marker, with a molecular weight of 200, 116, 97.2, 66.4, 44.3, 29, 20.1 and 14.3 kDa, respectively, from top to bottom.

FIG. 3 shows the enzymatic activity curve at different pH values. ♦ indicates enzymatic activity in NaAc buffer; ▪ indicates enzymatic activity in NaH₂PO₄ buffer; ▴ indicates enzymatic activity in Tris-HCl buffer.

FIG. 4 indicates pH tolerance of Xyl7. ♦ indicates enzymatic activity in NaH₂PO₄ buffer, pH 6.5; ▴ indicates enzymatic activity in NaH₂PO₄ buffer, pH 7.0; * indicates enzymatic activity in NaH₂PO₄ buffer supplemented with 70 mM mercaptoethanol, pH 7.0; x indicates enzymatic activity in NaH₂PO₄ buffer, pH 7.5.

FIG. 5 shows the enzymatic activity curve of Xyl7 under different temperatures.

FIG. 6 shows the tolerance of Xyl7 under different temperature. ♦ indicates enzymatic activity under 45° C.; ▪ indicates enzymatic activity under 50° C.; ▴ indicates enzymatic activity under 55° C.; x indicates enzymatic activity in NaH₂PO₄ buffer supplemented with 70 mM mercaptoethanol under 50° C.

FIG. 7 shows TLC analysis on the hydrolyzed substrates obtained by hydrolyzing birch xylan by Xyl7 under different conditions. 1: standard, wherein X1 is xylose, X2 is xylobiose, and X3 is xylotriose; 2: control (1% birch xylan that was not treated by the enzyme); 3: hydrolyzed product obtained after treatment for 10 minutes; 4: hydrolyzed product obtained after treatment for 1 hour; 5: hydrolyzed product obtained after treatment for 4 hours; 6: xylan was finally hydrolyzed to xylo-oligosaccharide after treating for 12 hours, which mainly includes xylobiose, xylotriose and xylotetraose.

FIG. 8 shows the residual enzymatic activity of Xyl7 tested after being placed for two hours under pH8 and pH9 at 55° C., 60° C. and 70° C., respectively.

FIG. 9 shows the relative residual activity of Xyl7 tested after being incubated under pH4 and pH5 at 37° C. for 15, 30, 45 and 60 minutes.

FIG. 10 shows the result obtained by the first round of screening on the random mutation library from directed evolution of Xyl7.

FIG. 11 shows the test result on the thermal stability of the mutants obtained from the first round of screening in the directed evolution.

FIG. 12 shows the result obtained from the second round of screening on the random mutation library and the screening result from saturation mutation at position 223.

FIG. 13 shows the test result on thermal stability of the mutants having combined mutation at position 223 at 55° C. (the upper panel) and 60° C. (the lower panel).

FIG. 14 shows the SDS-PAGE diagram obtained after expression of the fragment of Xyl7 consisting of residues 19-272 and purification of the expressed product. Lane 1: total proteins obtained after cell lysing; lane 2: precipitate from cell lysate; lane 3: supernatant of cell lysate; lane 4: 60 mM imidazole eluent; lane 5: 100 mM imidazole eluent; lane 6: 200 mM imidazole eluent; lane 7: 500 mM imidazole eluent; lane M: protein Marker, with a molecular weight of 200, 116, 97.2, 66.4, 44.3, 29, 20.1 and 14.3 kDa, respectively, from top to bottom.

FIG. 15 shows the test result on thermal stability of the mutants of the fragment of Xyl7 consisting of residues 19-272, which have mutation at position 223, at 55□ (the upper panel) and 60□ (the lower panel).

FIG. 16 shows the protein expression result of residue 19-267 of Xyl7, named Xyl7R2. Lane 1: total proteins obtained after cell lysing; lane 2: precipitate from cell lysate; lane 3: supernatant of cell lysate; lane 4: 60 mM imidazole eluent; lane 5: 100 mM imidazole eluent; lane 6: 200 mM imidazole eluent; lane 7: 500 mM imidazole eluent; lane M: protein Marker.

### SPECIFIC MODE FOR CARRYING OUT THE INVENTION

After a large scale of screening, the present inventors firstly isolated a novel xylanase, preferably, endo-1,4-β-xylanase, from the intestinal tract metagenome of termite, which exhibited high enzymatic activity, could be used in a wide range of temperature and pH, and had good application in industrial production. The amino acid sequence of the xylanase shows a highest similarity of 69% to the known amino acid sequences, demonstrating that it is a new protein. The xylanase of the present disclosure exhibits a very high enzymatic activity, with a specific activity of higher than 6340 U/mg at pH7.0 and 50□.

Aiming to overcome the defects present in the gene screening in traditional microbiology, metagenomics was developed. By directly extracting microbial nucleic acid from the environment and constructing metagenomic library (BAC, fosmid or plasmid library), defects caused by isolation and culture technology of microbe could be effectively overcome, thereby obtaining genetic information of all populations in the biocoenosis. The genetic information includes genes participating in bioconversion in the biocoenosis. Expression of the enzymes encoded by these genes in a clonal host could be used to screen various enzymes associated with bioconversion, thereby possibly obtaining a lot of new genes.

It is well known that xylanases of different natures are required in different applications, and xylanases of different natures may possibly be present in microbial genome in different ecological environments in the nature. Termite is an important organism which degrades lignocellulose in the natural ecosystem. Its symbiotic microbial biocoenosis in gut plays a key role in substance conversion of cellulose. Considering the high efficiency, uniqueness and complexity of the gut ecosystem of termite, termite was used as the system for screening xylanase by metagenomic technology in the present disclosure. By investigating the genes and enzymes from termite, we finally found the xylanase of the present disclosure.

The xylanase of the present disclosure can act on the interior of the long chain of xylan molecule, acting on the β-1,4-xylosidic linkage within the backbone of xylan to hydrolyze the large polyxylan to simple sugar, such as xylo-oligosaccharide.

As used herein, the term “the polypeptide of the present disclosure”, “the protein of the present disclosure”, “the xylanase of the present disclosure”, “Xyl7 protein”, “Xyl7 polypeptide” or “xylanase Xyl7” may be used interchangeable, all referring to the protein or polypeptide having the amino acid sequence (SEQ ID NO:2, its fragments or mutated forms or derivatives) of xylanase Xyl7. They include the xylanase Xyl7 containing or not containing the starting methionine.

As used herein, the terms “the gene of the present disclosure”, “xyl7 gene” and “xyl7” refer to the polynucleotide having the gene sequence encoding xylanase (SEQ ID NO:1, its mutated forms or derivatives).

As used herein, the “simple sugar” is generally a collective term of a kind of sugars formed after cleaving the xylan chain, which have a chain length less than that before cleavage. For example, the simple sugar contains 1-50 xyloses, preferably, 1-30 xyloses, more preferably, 1-15 xyloses, more preferably, 1-10 xyloses, such as 2, 3, 4, 5, 6, 7, 8, 9 xyloses. The simple sugar includes xylo-oligosaccharide, xylobiose, xylotriose, xylotetraose, etc. In the present disclosure, the simple sugar also refers to xylo-oligosaccharide or small-amount xyloses.

As used herein, the “xylose” refers to a monosaccharide containing 5 carbon atoms, with a molecular formula of C₄H₉O₄CHO. The “xylan” is a polymer of “xylose”.

As used herein, “isolated” refers to that a substance is isolated from its original environment (if it is a natural substance, the original environment is the natural environment). For example, the polynucleotide and polypeptide in a natural state within a living cell are not isolated or purified. However, if the polynucleotide or polypeptide is separated from other substances simultaneously present in the natural state, it is isolated and purified.

As used herein, the “isolated Xyl7 protein or polypeptide” refers to that the Xyl7 polypeptide is essentially free of other proteins, lipids, sugars or other substances that are naturally associated with the polypeptide. The skilled artisan can use the standard protein purification technology to purify Xyl7 protein. The substantially pure polypeptide could produce a single main band on the non-reduced polyacrylamide gel. The purity of Xyl7 polypeptide can be used for analysis of amino acid sequence.

The polypeptide of the present disclosure may be a recombinant polypeptide, a natural polypeptide, or a synthetic polypeptide, preferably a recombinant polypeptide. The polypeptide of the present disclosure may be a natural, purified product, or may be chemically synthesized product, or may be produced from prokaryotic host or eukaryotic host, such as bacterium, yeast, higher plant, insect and mammal cell, by recombinant technology. According to the host used in the recombinant production protocol, the polypeptide of the present disclosure may be glycosylated, or non-glycosylated. The polypeptide of the present disclosure may also include or not include the starting methionine residue.

The present disclosure also includes the fragments, derivatives and analogs of the Xyl7 protein. As used herein, the terms “fragment”, “derivative” and “analog” refer to the polypeptide substantially retaining the same biological function or activity as that of the native Xyl7 protein. The fragment, derivative or analog of the polypeptide of the present disclosure may be (i) a polypeptide with one or more conservative or non-conservative amino acid residue(s), preferably conservative amino acid residue, being substituted, wherein the substituted amino acid residue may or may not be encoded by genetic codes; or (ii) a polypeptide with one or more amino acid residue(s) being substituted by a substituent group, or (iii) a polypeptide formed by fusing the mature polypeptide with another compound, such as the compound for extending the half life of the polypeptide, such as polyethylene glycol, or (iv) a polypeptide formed by fusing an additional amino acid sequence, such as a leader sequence, a secretion sequence, a sequence for purifying the present polypeptide, or proteinogen sequence, or an IgG fragment of an antigen, with the present polypeptide. According to the present disclosure, these fragments, derivatives and analogs fall within the scope known to the skilled in the art.

In the present disclosure, the term “Xyl7 polypeptide” refers to the polypeptide set for in SEQ ID NO:2, or active fragments and active derivatives thereof, which have Xyl7 protein activity. In a preferred embodiment, the active fragment may be a fragment containing the conservative domain of SEQ ID NO:2 (amino acids 32-256). For example, the fragment may be a fragment containing amino acid residues 19-267 of SEQ ID NO:2. In other embodiments, the active fragment may be a fragment containing amino acid residues 19-272 of SEQ ID NO:2. For example, the fragment may be amino acids 19-450, 19-300, etc., of SEQ ID NO:2, preferably amino acids 19-267 and 19-272 of SEQ ID NO:2. Additionally, for example, mutation can be present outside the conservative domain of SEQ ID NO:2 (amino acids 32-256). In a preferred embodiment, mutation is taken place outside, such as, amino acid residue 19 to amino acid residue 267 or 272 of SEQ ID NO:2. Mutation can be 1-20, such as 1-10, preferably 1-5 or 1-3 deletion, substitution and insertion of amino acid. The term also includes the mutated forms of SEQ ID NO: 2 or amino acids 19-272 or 19-267 of SEQ ID NO:2, which have the same function as Xyl7 protein. These mutated forms include, but is not limited to, deletion, insertion and/or substitution of one or more (generally 1-50, preferably 1-30, more preferably 1-20, more preferably 1-10, most preferably 1-5) amino acids, and addition or deletion of one or more (generally less than 20, preferably less than 10, more preferably less than 5) amino acids at the C terminus and/or N terminus. For example, in the field of the art, substitution with amino acid having close or similar property generally will not change the function of protein. For example, addition or deletion of one or more amino acids at the C terminus and/or N terminus generally will not change the function of protein. Additionally, for example, the same catalytic function as that of the intact protein can also be obtained even only the catalytic domain of the protein is expressed while the carbohydrate binding domain is not expressed. The mutated forms of the polypeptide include homologous sequence, conservative mutant, allelic variant, native variant, induced variant, protein encoded by a DNA that can hybridize to xyl7 DNA under high or low stringent condition, and polypeptide or protein obtained by utilizing an antibody against the Xyl7 polypeptide. The present disclosure also provides other polypeptides, such as a fusion protein comprising the Xyl7 polypeptide or its fragment. In additional to the almost full length polypeptide, the present disclosure also includes the soluble fragments of the Xyl7 polypeptide. Generally, the fragment contains at least about 10 continuous amino acids, generally at least 30 continuous amino acids, preferably at least 50 continuous amino acids, more preferably at least 80 continuous amino acids, most preferably at least 100 continuous amino acids, of the Xyl7 polypeptide sequence.

The mutated forms of SEQ ID NO:2 or amino acids 19-272 or 19-267 of SEQ ID NO:2 of the present disclosure include, but is not limited to, substitution mutation occurred at one or more positions selected from the group consisting of 32, 37, 42, 80, 205, 219, 221, 222, 223, 228 and 386 of SEQ ID NO:2, or at one or more positions corresponding to the following amino acid residues of SEQ ID NO:2: 32, 37, 42, 80, 205, 219, 221, 222, 223, 228 and 386. Amino acid used for substitution is not specially limited. In some embodiments, the mutated sequence of the present disclosure may contain one or more substitutions selected from the group consisting of K32T, N37D, S42N, M80I, K205E, E219D, A221T, M222L, K223M, K223E, K223T, K223C, K223S, K223G, K223L, T228S, and A386S.

In some embodiments, the mutated forms of the present disclosure include, but are not limited to, the sequences set for in SEQ ID NO: 13, 15, 17, 19, 21, 23, 25, 27 and 29. The present disclosure also includes the coding sequences of these mutated polypeptides, which, for example, are the sequences set forth in SEQ ID NO:12, 14, 16, 18, 20, 22, 24, 26 and 28.

Also provided in the present disclosure are analogs of the Xyl7 protein or polypeptide. The difference between these analogs and the native Xyl7 polypeptide may be the difference in amino acid sequence or in modification that does not affect the sequence, or both. These polypeptides include native or induced genetic variants. Induced variants may be obtained via various technologies, such as via radiation or exposure to mutagen to produce random mutation or via site-directed mutagenesis or other known molecular biological technique. Analogs further include those having a residue different from the native L-amino acid, such as a D-amino acid, and an amino acid that is not naturally occurring or that is synthetic, such as β, γ-amino acid. It should be understood that the polypeptide of the present disclosure is not limited to the above representative polypeptides listed above. Modification that generally does not change the primary structure includes in vivo or in vitro chemical derivative forms of the polypeptide, such as acetylation or carboxylation. Modification further includes glycosylation, such as the polypeptides produced by glycosylation modification during its synthesis or processing or further processing. This kind of modification can be accomplished by exposing the polypeptide to the enzyme used for glycosylation, such as the mammal glycosylase or deglycosylase. Modified forms further comprise sequences with phosphorylated amino acid residue, such as phosphotyrosine, phosphoserine and phosphothreonine. Also included are polypeptides that are modified to increase its anti-proteolytic property or to optimize solubility.

In the present disclosure, the “conservative variant polypeptides of Xyl7 protein” refers to polypeptides with at most 20, preferably at most 10, more preferably at most 5, most preferably at most 3 amino acids being replaced by amino acids having close or similar amino acids, as compared to SEQ ID NO:2 or amino acids 19-267 or 19-272 of SEQ ID NO:2. These conservative variant polypeptides preferably are produced according to the amino acid replacement indicated in Table 1.

TABLE 1

| Initial Residue | Representative Substitution | Preferred Substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The amino terminus or carboxyl terminus of the present Xyl7 protein may contain one or more polypeptide fragment(s) as a protein tag. Any suitable tag can be used in the present disclosure. For example, the tag may be FLAG, HA, HAL c-Myc, Poly-His, Poly-Arg, Strep-TagII, AU1, EE, T7, 4A6, E, B, gE and Ty1. These tags may be used in protein purification. Some tags and their sequences are listed in Table 2.

TABLE 2

| Tag | Number of Residue | Sequence | SEQ ID NO: |
|---|---|---|---|
| Poly-Arg | 5-6, generally 5 | RRRRR | 5 |
| Poly-His | 2-10, generally 6 | HHHHHH | 6 |
| FLAG | 8 | DYKDDDDK | 7 |
| Strep-TagII | 8 | WSHPQFEK | 8 |
| C-myc | 10 | WQKLISEEDL | 9 |

To allow the translated protein to be secretorily expressed, such as secreted outside the cell, a signal peptide sequence, such as the pelB signal peptide, etc., may be added at the terminus of the Xyl7. The signal peptide may be cleaved during secretion of the polypeptide from the cell.

The polynucleotide of the present disclosure may be in a DNA or RNA form. The DNA form includes cDNA, genomic DNA or artificially synthetic DNA. DNA may be single-stranded or double-stranded. DNA may be a coding strand or a non-coding strand. The coding sequence that encodes the mature polypeptide may be identical to the coding sequence set forth in SEQ ID NO:1 or its degenerate variant. As used herein, the “degenerate variant” in the present disclosure refers to the nucleic acid sequence encoding the protein of SEQ ID NO:2 but having differences in coding sequence from the coding sequence shown in SEQ ID NO:1.

Polynucleotide encoding the mature polypeptide of SEQ ID NO:2 includes the coding sequence that only encodes the mature polypeptide; the coding sequence of the mature polypeptide and various additional coding sequences; the coding sequence of the mature polypeptide (and optionally additional coding sequences) and non-coding sequence.

The term “polynucleotide encoding polypeptide” may be a polynucleotide comprising a polynucleotide encoding the present polypeptide, or further comprising additional coding sequence and/or non-coding sequence.

Also contemplated in the present disclosure are variants of the above polynucleotide, which encode polypeptides having the same amino acid sequence as that of the present disclosure, or fragments, analogs and derivatives thereof. The variants of the polynucleotide may be a naturally occurring allelic variant or non-naturally occurring variant. These nucleotide variants include substitution variant, deletion variant and insertion variant. As known in the art, the allelic variant is an alternative of polynucleotide, which may contain substitution, deletion or insertion of one or more nucleotide(s) but the function of the encoded polypeptide will not be substantively changed.

The present disclosure also relates to the polynucleotides hybridized to the above sequence and having at least 50%, preferably at least 70%, more preferably at least 80% sequence identity between two sequences. Specifically, the present disclosure relates to the polynucleotides hybridizable to the polynucleotide of the present disclosure under stringent condition. In the present disclosure, the “stringent condition” refers to (1) hybridizing and eluting at a relatively low ionic strength and a relatively high temperature, such as 0.2×SSC, 0.1% SDS, 60° C.; or (2) hybridizing in the presence of a denaturant, such as 50% (v/v) formamide, 0.1% calf serum/0.1% Ficoll, 42° C., etc.; or (3) hybridizing only when two sequences having an identity of at least 90%, preferably 95% or more. Additionally, the polypeptide encoded by the hybridizable polynucleotide exhibit the same biological function and activity as the mature polypeptide set forth in SEQ ID NO:2 or amino acids 19-267 of SEQ ID NO:2.

The present disclosure also relates to nucleic acid fragment that hybridizes to the above sequence. As used herein, the “nucleic acid fragment” contains at least 15 nucleotides, preferably at least 30, more preferably at least 50, most preferably at least 100 nucleotides. The nucleic acid fragment may be used for amplifying nucleic acid, such as PCR, to determine and/or isolate the polynucleotide encoding Xyl7 protein.

Preferably, the present polypeptide and polynucleotide are provided in an isolated form, more preferably, purified to a homogeneous state.

The full length xyl7 nucleotide sequence or its fragment may be obtained by PCR amplification, recombinant method or artificial synthesis. For the PCR amplification, sequences of interest can be obtained by amplification by using primers designed according to the related nucleotide sequence disclosed in the present disclosure, especially the sequence of the open reading frame, and the commercially available cDNA library or a cDNA library prepared according to the conventional method known in the art as template. When the sequence is too long, two or multiple PCR amplification should be done and then the fragments obtained from each amplification can be linked together in proper order.

Once a sequence of interest is obtained, a large amount of the sequences can be produced through a recombinant method. Generally, the sequence is cloned into a vector and then the vector is transferred into a cell. Sequence of interest could then be isolated from the proliferative host cell via a conventional method.

Additionally, the sequence of interest can be artificially synthesized, especially when it is a short fragment. Generally, many small fragments are firstly synthesized and then they are linked together to obtain a long fragment.

Currently, the DNA sequences encoding the present proteins, or their fragments or derivatives could be completely obtained through chemical synthesis. The DNA sequence can then be introduced into various existing DNA molecules, such as vectors, and cells known in the art. Additionally, mutation can be introduced into the protein sequence of the present disclosure via chemical synthesis.

Preferably, method for amplification of DNA/RNA by PCR technique is used to obtain the genes of the present disclosure. Especially in the case that it is difficult to obtain the full length cDNA from a library, RACE (RACE-cDNA, rapid amplification of cDNA ends) is preferred. Primers used in PCR may be suitably selected according to the sequence information disclosed in the present disclosure and synthesized by the conventional method. The amplified DNA/RNA fragment could be isolated and purified by a conventional method, such as gel electrophoresis.

The present disclosure also relates to vectors containing the present polynucleotide, host cells genetically engineered by the vector or coding sequence of Xyl7 protein of the present disclosure, and methods for producing the polypeptide of the present disclosure via a recombinant technique.

The recombinant Xyl7 polypeptide may be expressed or produced by utilizing the polynucleotide sequence of the present disclosure via a conventional recombinant DNA technique. Generally, the following steps are included:

(1) Transforming or transfecting suitable host cells with the present polynucleotide (or variants thereof) encoding the Xyl7 polypeptide or the recombinant expression vector containing the polynucleotide;

(2) Culturing the host cells in a suitable culture medium;

(3) Isolating and purifying proteins from the culture medium or cells.

In the present disclosure, the xyl7 polynucleotide sequence may be inserted into a recombinant expression vector. The term “recombinant expression vector” refers to the bacterial plasmid, bacteriophage, yeast plasmid, plant cell virus, mammalian cell virus such as adenovirus, retrovirus, or other vectors known in the art. Any plasmid and vector can be used as long as they can replicate and stabilize in the host. One important feature of an expression vector is that it generally contains a replication origin, a promoter, a marker gene and a translation control element.

Methods well known by the skilled in the art can be used to construct an expression vector that contains DNA sequence encoding Xyl7 and suitable transcription/translation control signal. These methods include in vitro recombinant DNA technique, DNA synthesis technique, in vivo recombinant technique, etc. The DNA sequence may be effectively linked to a suitable promoter in the expression vector to direct mRNA synthesis. Representative examples of promoter include lac or trp promoter from E. coli, PL promoter from bacteriophage λ; eukaryotic promoter, including CMV immediate early promoter, HSV thymidine kinase promoter, early and late SV40 promoter, LTRs of retrovirus, and some other promoters known to be able to control expression of a gene in prokaryotic or eukaryotic cell or its virus. The expression vector further comprises ribosome bind site for initiating translation and transcription terminator.

Additionally, the expression vector preferably contains one or more selectable marker gene(s) for providing phenotypic characteristics for the transformed host cell, such as dihydrofolate reductase, neomycin resistance gene and green fluorescent protein (GFP) used in culture of eukaryotic cell, or tetracycline or ampicillin resistance gene used for E. coli.

Vector containing the above suitable DNA sequence and suitable promoter or control sequence can be used to transform a suitable host cell to allow it to express the protein.

Host cell may be prokaryotic cell, such as bacterial cell; or lower eukaryotic cell, such as yeast cell; or a higher eukaryotic cell, such as mammal cell. Representative examples include E. coli, Streptomyces; cells from Salmonella typhimurium; fungal cell, such as yeast; plant cell; insect cell, such as Drosophila melanogaster S2, or Sf9; animal cell, such as CHO, COS, 293 cell, or Bowes melanoma cells.

When expressing the polynucleotide of the present disclosure in a higher eukaryotic cell, insertion of an enhancer sequence in the vector will improve the transcription. Enhancer may be a cis-acting factor of DNA, generally containing 10 to 300 bps, which acts on the promoter to improve the gene transcription. Examples of enhancer include SV40 enhancer, which contain 100 to 270 bps and locate at the later side of the replication origin; polyoma virus enhancer and adenovirus enhancer, which locate at the later side of the replication origin, etc.

It is well known for the skilled in the art to select suitable vector, promoter, enhancer and host cell.

Transformation of host cell with recombinant DNA can be performed through the conventional technique known in the art. When the host is a prokaryotic organism, such as E. coli, competent cells that can adsorb DNA can be harvested after exponential phase and then treated by CaCl₂ method. All steps are known in the art. MgCl₂ may be used in another method. When necessary, transformation can be performed by electroporation. When the host is a eukaryotic organism, the following DNA transfection methods can be used: calcium phosphate coprecipitation method and conventional mechanical method, such as microinjection, electroporation, and liposome packaging, etc.

The resultant transformant may be cultured by a conventional method to express the polypeptide encoded by the present gene. According to the used host cell, the culture medium used for culture may be selected from various conventional culture mediums. Cultivation is performed under conditions suitable for growth of host cell. When the host cells grow and reach to a suitable cell density, the selected promoter is induced by a suitable method, such as temperature conversion or chemical induction, and the cells are further cultured for a period of time.

In the above-mentioned methods, the recombinant polypeptide may be expressed within the cell, on the cell membrane, or secreted outside the cell. When necessary, the recombinant protein may be isolated and purified via various isolation methods by utilizing its physical, chemical or other properties. These methods are known to the skilled artisan. Examples of these methods include, but are not limited to, conventional renaturation treatment, treatment by protein precipitant (salting out), centrifugation, breakage of bacterium through osmosis, super processing, super centrifugation, molecular screen chromatography (gel filtration), adsorption chromatography, ion exchange chromatography, high-performance liquid chromatography (HPLC) and other liquid chromatography techniques, and combination thereof.

The use of the recombinant Xyl7 includes, but is not limited to, hydrolyzing xylan, cleaving the long xylan chain into a short chain, or forming simple sugar. Most known xylanases exhibit an activity lower than the activity of the present Xyl7 enzyme. It is expected that the enzymatic activity of Xyl7, or its applicable pH range, temperature range and thermal stability may be further improved by modification of its protein molecule. Thus, Xyl7 has a promising application prospect. Some techniques for modifying protein are well known to the skilled in the art. Thus, xylanases formed by modifying Xyl7 by these techniques are also contemplated in the present disclosure.

The expressed recombinant Xyl7 protein is used to screen a library of polypeptide, which may find out polypeptide molecules that may have therapeutic value and can inhibit or stimulate the function of the Xyl7 protein.

In another aspect, the present disclosure also comprises polyclonal and monoclonal antibodies, especially monoclonal antibodies, specifically against the polypeptide encoded by Xyl7 DNA or its fragment. As used herein, “specificity” refers to that antibody can bind to the product of Xyl7 gene or its fragment. Preferably, it refers to that the antibody can bind to the product of xyl7 gene or its fragment but does not recognize or bind to the other irrelevant antigen molecules. In the present disclosure, antibody comprises the molecules which can bind to and inhibit the Xyl7 protein and those not affecting the function of the Xyl7 protein. The present disclosure also comprises the antibodies which can bind to the modified or un-modified product of the Xyl7 gene.

The antibody of the present disclosure may be prepared by various techniques known to the skilled artisan. For example, the purified product of the Xyl7 gene or its antigenic fragment may be administered to animal to induce production of polyclonal antibody. Similarly, cell expressing the Xyl7 protein or its antigenic fragment may be used to immunize animal to produce the antibody. The antibody of the present disclosure may be a monoclonal antibody. Such monoclonal antibody may be prepared by hybridoma technique (see Kohler et al., Nature 256; 495, 1975; Kohler et al., Eur. J. Immunol. 6: 511, 1976; Kohler et al., Eur. J. Immunol. 6: 292, 1976; Hammerling et al., In Monoclonal Antibodies and T Cell Hybridomas, Elsevier, N. Y., 1981). Antibody against the Xyl7 protein may be used to detect the Xyl7 protein in a sample.

Substances that interact with Xyl7 protein, such as inhibitor, agonist or antagonist, etc., may be screened via various conventional screening methods by utilizing the present protein.

The present disclosure may also provide a composition, comprising an effective amount of the present Xyl7 polypeptide and a bromatologically acceptable or industrially acceptable carrier. Such carrier includes, but is not limited to, water, buffer, glucose, water, glycerol, ethanol, and combination thereof. The skilled in the art can determine the effective amount of the Xyl7 polypeptide in the composition according to the actual use of the composition.

Substances for regulating the activity of the present Xyl7 enzyme may be added into the composition. Any substances capable of improving the enzymatic activity can be used. Preferably, substance which improves the activity of the present Xyl7 enzyme is selected from the group consisting of K⁺, Mn²⁺, or materials that could be hydrolyzed to form K⁺ or Mn²⁺ after adding to the substrate, such as KCl and manganese sulfate. Additionally, some substances may decrease the enzymatic activity, which is selected from the group consisting of Ni²⁺, Zn²⁺, Fe³⁺ and EDTA, or materials that could be hydrolyzed to form Ni²⁺, Zn²⁺ or Fe³⁺ after adding to the substrate.

After obtaining the present Xyl7 enzyme, the skilled artisan can conveniently use the enzyme to hydrolyze substrate, especially xylan, according to the teaching of the present disclosure. As a preferred embodiment of the present disclosure, a method for forming simple sugar are provided, which comprises treating the substrate to be hydrolyzed with the present Xyl7 enzyme, wherein the substrate includes birch xylan and beech xylan, etc. Generally, the substrate to be hydrolyzed is treated by the Xyl7 enzyme under pH 3.5-10. Generally, the substrate to be hydrolyzed is treated by the Xyl7 enzyme under 30-80° C. Preferably, K⁺, Mn²⁺, or materials that could be hydrolyzed to form K⁺ or Mn²⁺ after adding to the substrate is added during treating with the Xyl7 enzyme.

In one embodiment of the present disclosure, an isolated polynucleotide is provided, which encodes a polypeptide having an amino acid sequence of SEQ ID NO:2. The polynucleotide of the present disclosure is isolated from the Fosmid library constructed from the gut system of termite. Its sequence is set forth in SEQ ID NO:1, with a full length of 1518 bases. It encodes the Xyl7 protein (SEQ ID NO:2) with a full length of 505 amino acids. In the sequence of Xyl7 protein (SEQ ID NO:2), amino acids 32-256, from the amino terminus, is the conservative domain of the Glycosyl Hydrolase Family 11. The Xyl7 protein has a similarity of 69% to the known amino acid sequences, demonstrating that it is a new endo-1,4-β-xylanase.

As demonstrated by experiments, the present endo-1,4-β-xylanase exhibit a very high xylanase activity, a wide applicable pH range and wide applicable temperature range. Thus, it has a promising application prospect.

The present disclosure also provides a polypeptide having an improved thermal stability, which is derived from the original polypeptide and exhibits xylanase activity and significantly improved thermal stability. The original polypeptide is preferably the polypeptide the amino acid sequence of which is set forth in SEQ ID NO:2 or its fragment that comprising amino acids 19-267 or 19-272. Preferably, the polypeptide having an improved thermal stability at least contains a substitution mutation at the position corresponding to amino acid residue 32 or 223 of SEQ ID NO:2. Preferred mutations include K32T, K223E, K223C, K223S, or combination thereof. More preferably, the mutation is a combination of K32T with K223C, or a combination of K32T with K223S.

Also provided in the present disclosure is a method for increasing the thermal stability of a polypeptide, comprising mutating the amino acid of the xylanase polypeptide at the position corresponding to amino acid residue 32 and/or 223 of SEQ ID NO:2 to obtain a xylanase having an improved thermal stability. The xylanase may be other xylanases known in the art. In some embodiments, the xylanase is the xylanase disclosed in the subject application. Specifically, the method comprises mutating the amino acid at amino acid residue 32 or 223 of SEQ ID NO:2 or its active fragments.

As used herein, improvement of thermal stability is intended to mean that the reduction degree of activity of a polypeptide is lower than that of the wild type polypeptide (the starting polypeptide) after treating at a temperature, such as 15-90° C., preferably 30-60° C., more preferably 50-55° C., such as 50° C. or 55° C. a period of time, as compared to the activity before treating at that temperature. In other words, the activity retained by the polypeptide after treatment is relatively high. Substrate used in the treatment for thermal stability may be the specific substrate of the xylanase. In the present disclosure, the substrate may be, for example, birch xylan and beech xylan. The pH value used in the treatment is generally in a range of 3-12, preferably 5.5-10, more preferably about 7.0.

Additionally, it should be understood that the above polypeptides having an improved thermal stability and the method for increasing the thermal stability of a polypeptide may be modified by the skilled artisan within a certain range. Modifications, such as addition, reduction or deletion of amino acid in addition to mutation at amino acid residue 32 or 223 of SEQ ID NO:2, or addition of a signal peptide, do not affect the claims of the present disclosure. If the modifications relate to amino acid at position corresponding to amino acid residue 32 or 223 of SEQ ID NO:2, then the modifications shall fall within the scope of the subject description and claims.

The invention will be further illustrated by making reference to the following specific examples. It should be understood that these examples are only for illustrating the invention, but not for limiting the scope of the invention. For the experiments the specific conditions of which are not specifically indicated, they generally were performed according to the conditions described in Sambrook, et al., Molecular Cloning: A Laboratory Manual (New York: Cold Spring Harbor Laboratory Press, 1989), or according to the conditions recommended by the manufacturer. Unless otherwise indicated, the percentage and part are calculated by weight.

Unless otherwise defined, all professional and scientific terms used herein have the same meanings well known to the skilled artisan. Additionally, any method and material similar to or equivalent to the contents disclosed herein can be used in the present disclosure. The preferred embodiments and materials described herein are merely for exemplary purpose.

### Example 1

### Isolation of Endo-1,4-β-Xylanase and its Coding Gene

The positive clone of xylanase was screened from the metagenomics library of the microorganisms in termite gut by utilizing metagenomic technique through a conventional method. The plasmid DNA of the clone was extracted and subjected to 454 high-throughput sequencing. The sequences were ligated to obtain a complete Fosmid sequence. ORF was found by DNAStar software. GenBank database was searched by BlastP of NCBI (http://www.ncbi.nlm.nih.gov) to obtain a new gene encoding endo-1,4-β-xylanase, which had the nucleotide sequence set forth in SEQ ID NO:1, named Xyl7. Nucleotides 1-1518 from the 5′ end of SEQ ID NO:1 is the open reading frame (ORF) of Xyl7. Nucleotides 1-3 from the 5′ end of SEQ ID NO:1 is the starting codon ATG of the Xyl7 gene, and nucleotides 1516-1518 from the 5′ end of SEQ ID NO:1 is the termination codon TAA of the Xyl7 gene.

The Xyl7 gene of endo-1,4-β-xylanase encodes a protein Xyl7 containing 505 amino acids, which contains the amino acid sequence set forth in SEQ ID NO:2. The theoretical molecular weight of the protein, as predicted by software, is 51.9 kDa and the isoelectric point (pI) is 8.87. Amino acids 32-256 from the amino terminus of SEQ ID NO:2 is the conservative domain of the Glycosyl Hydrolase Family 11(GH 11).

After identification, Xyl7 could act on the internal β-1,4-xylosidic linkage of the backbone of birch xylan or beech xylan in an endo-cleavage and highly efficient manner. When the action time was relatively short (10 minutes), the large polyxylan was preliminarily hydrolyzed to oligoxylan and when the action time was relatively long (12 hours), the polyxylan was finally hydrolyzed to xylo-oligosaccharide, mainly including xylobiose, xylotriose and xylotetraose.

Xyl7 showed a highest homology of 69% to the endo-xylanase having known sequence after comparison, demonstrating that it was a new endo-1,4-β-xylanase.

### Example 2

### Expression of Xyl7 in E. coli

1. Construction of Recombinant Expression Vector

The predicted ORF coding gene of endo-1,4-β-xylanase was amplified by PCR from the screened xylanase positive clone. The forward primer was 5′ GAGACTCCATATGCAAGGTCCCACATGGACT 3′ (SEQ ID NO: 3), with Nde I recognition site (CATATG) added at its 5′ end. The reverse primer was 5′ CGGAATTCTTACCTCACCATAACCCT 3′ (SEQ ID NO: 4), with EcoR I recognition site (GAATTC) added at its 5′ end.

The PCR product was purified and digested by Nde I and EcoR I enzymes. The DNA fragment obtained after enzyme digestion was recovered by Axgen PCR Product Column Recovery Kit and ligated with pET-28a vector (Novagen) recovered after digested overnight by the same two enzymes by T4 DNA ligase at 16□ to produce the recombinant expression vector pET 28a-Xyl7. The N terminus of the expressed product had a His tag (6×His-Tag) provided by the expression vector, which facilitated the subsequent purification.

2. Expression of the Xyl7 Gene in E. coli BL21(DE3) and Purification of the Expressed Product

(1) Expression of Xyl7

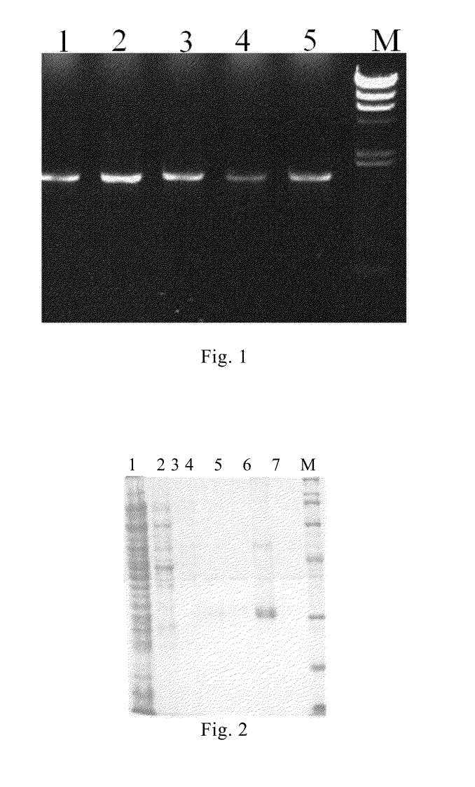

The above-constructed plasmid pET 28a Xyl7 was transformed into E. coli BL21(DE3). Five monoclones were picked up from the resultant BL21(DE3)/pET28a-Xyl7 transformants and inoculated into LB culture liquid containing ampicillin. After culturing in a shaker for a short period, the positive clones were identified by PCR by directly using the bacterial liquid as template and by using the T7 promoter primer (cat. no. 69348-3) and T7 terminator primer (cat. no. 69337-3) for amplifying the vector. Results were shown in FIG. 1, in which target fragments were amplified from all five monoclones.

E. coli BL21 (DE3)/pET28a-Xyl7 was inoculated in LB culture liquid containing 100 μg/ml ampicillin and cultured overnight at 37° C., 200 rpm. One milliliter of culture liquid was inoculated to 100 ml LB culture liquid and cultured at 37° C., 200 rpm until (Moo reached 0.6-0.8. After cooling, 80 μM IPTG were added and cultivation was continued at 24° C., 200 rpm for 16 hours. Thallus was recovered by centrifugation. The recovered thallus was suspended by a lysis buffer (NaH₂PO₄ 50 mmol/L, NaCl 300 mmol/L, pH7.4). After breaking the cell by ultrasonic wave, the supernatant was collected by centrifugation, which was a crude enzyme solution.

Ni-NTA Column (Qiagen) was used to purify the crude enzyme solution. The wash buffer used during purification contained NaH₂PO₄ 50 mmol/L, NaCl 300 mmol/L, pH7.0. The elution buffer containing different concentrations of imidazole (20, 40, 60, 100, 200 and 500) contained NaH₂PO₄ 50 mmol/L, NaCl 300 mmol/L, imidazole 20-500 mmol/L, pH 7.0. The protein SDS-PAGE electrophoresis was performed by using 5 μl elution solutions. Results were shown in FIG. 2, in which lane 1 indicated the supernatant of cell lysate; lane 2 indicated 20 mM imidazole eluent; lane 3 indicated 40 mM imidazole eluent; lane 4 indicated 60 mM imidazole eluent; lane 5 indicated 100 mM imidazole eluent; lane 6 indicated 200 mM imidazole eluent; and lane 7 indicated 500 mM imidazole eluent. From FIG. 2, the target protein was eluted in a great amount when eluted by 200 mM imidazole and a single bank was observed after electrophoresis, indicating that target protein of high purity was obtained. All eluents containing the target protein were pooled and subjected to concentration and dialysis by vivaspin 6 ultrafiltration tube from GE (10 Kd interception value) and at the same time a replacement buffer containing 20 mM NaH₂PO₄, pH 7.4 was used to remove imidazole.

### Example 3

### Analysis on the Zymological Property of the Recombinant Xyl7 Protein

Enzymatic activity of the endo-1,4-β-xylanase was tested by a DNS method as follows:

(1) Preparation of DNS

10 g NaOH were weighed and dissolved in about 400 ml ddH₂O. 10 g dinitrosalicylic acid, 2 g phenol, 0.5 g anhydrous sodium sulfite and 200 g potassium sodium tartrate tetrahydrate were weighed and dissolved in about 300 ml ddH₂O. The two solutions were mixed, diluted to 1 liter and preserved in dark.

(2) Preparation of Standard Curve

Nine thin-wall centrifuge tubes were loaded with the xylose standard sample prepared according to the xylose stock volume and pure water volume described in Table 3. The concentration of the xylose stock was 10 mg/ml.

TABLE 3

| | Number of standard sample | | | | | | | | |
|---|---|---|---|---|---|---|---|---|---|
| | 1 | 2 | 3 | 4 | 5 | 6 | 7 | 8 | 9 |
| Content of xylose | 0 | 10 | 20 | 30 | 40 | 70 | 80 | 120 | 150 |
| (μg) | | | | | | | | | |
| Volume of xylose | 0 | 1 | 2 | 3 | 4 | 7 | 8 | 12 | 15 |
| stock (μl) | | | | | | | | | |
| Volume of pure | 100 | 99 | 98 | 97 | 96 | 93 | 92 | 88 | 85 |
| water (μl) | | | | | | | | | |

100 μl DNS were added into each of the above standard samples. The mixtures were subjected to boiled water bath for 5 minutes for developing color. Absorbance value at 540 nm was detected by a microplate reader. Standard sample 1 was a blank control. The absorbance value of the blank control was subtracted from that of each sample and then the standard curve was plotted.

(3) Detection of Standard Enzymatic Activity

In a 100 μl reaction system, birch xylan was added to a final concentration of 1% (w/w), and Na₂HPO₄/NaH₂PO₄ buffer (pH7.0) was added to a final concentration of 100 mM. A suitable amount of enzyme solution diluted by the abovementioned buffer was added to the reaction system for reaction at 50□ for 10 minutes. 100 μl DNS were added to stop the reaction. For the control, 100 μl DNS were firstly added to the above reaction system before adding the enzyme solution. The reaction mixtures were subjected to a boiled water bath for 5 minutes to develop color. Absorbance value at 540 nm was detected by a microplate reader. After subtracting the absorbance value of the control from that of the sample, the enzymatic activity unit (U) was calculated based on the standard curve.

Definition of enzymatic activity unit (U): 1U refers to the amount of enzyme required for catalytically hydrolyzing xylan to produce 1 μmol xylose.

Definition of specific activity unit: enzymatic activity of 1 mg protein (U/mg).

Results showed that the specific activity of Xyl7 on beech xylan was 6340 U/mg at pH7.0, 50° C.

(4) Detection of the Optimum pH of Xyl7

Buffers having different pH values in the range of 3.5-10 in a gradient of 0.5 unit were prepared, wherein NaAc with a final concentration of 100 mM was used to prepare the buffers having a pH in the range of 3.5-6.0, Na₂HPO₄/NaH₂PO₄ with a final concentration of 100 mM was used to prepare the buffers having a pH in the range of 6.0-8.0, and Tris-HCl with a final concentration of 100 mM was used to prepare the buffers having a pH in the range of 8.0-10.0. The enzyme solution was added into each pH buffer system and the enzymatic activity was tested according to the above standard enzymatic activity detection steps. Xyl7 exhibited a highest specific activity in the Na₂HPO₄/NaH₂PO₄ buffer (pH7.0) at 50□. This specific activity value was used as a reference value and its relative activity was defined as 100%. The relative activity of the enzyme obtained at each pH value was a ratio between the specific activity of the enzyme at each pH value and the reference value.

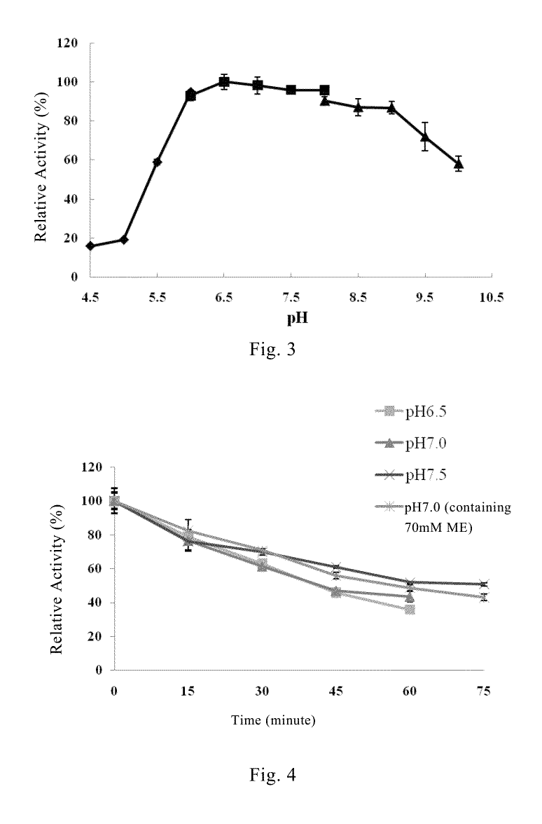

Results were shown in FIG. 3. The optimum pH of Xyl7 was 7.0. Xyl7 exhibited a relative activity of 50% or more in the pH range of from 5.5 to 10, indicating that Xyl7 exhibited a relatively broad reaction pH range and could be applicable in a wide range of pH value.

(5) Detection on pH Tolerance of Xyl7

The enzyme solution was kept in buffers of different pH values (6.5, 7.0 and 7.5) or at pH 7.0 with addition of 70 mM mercaptoethanol at 50° C. for different times (15 min, 30 min, 45 min, 60 min, 75 min). Enzymatic activity was tested according to the above standard enzymatic activity detection steps (enzymatic activity was tested after reacting at pH7.0, 50° C. for 10 minutes). The specific activity value of Xyl7 obtained after storing at pH7.0, 50° C. for 0 minute and reacting at 50° C. for 10 minutes was used as a reference value, and its relative activity was defined as 100%. The relative activity of the enzyme obtained after storing in each buffer having different pH values for different times was a ratio between the specific activity value of the enzyme subjected to above treatment and the reference value.

Results were shown in FIG. 4. Xyl7 could tolerate a wide range of pH values. After storing in buffer having a pH of 6.5, 7.0 or 7.5 for 45 min, the enzyme, in all cases, retained 50% or more of the highest activity.

(6) Detection on the Optimum Temperature of Xyl7

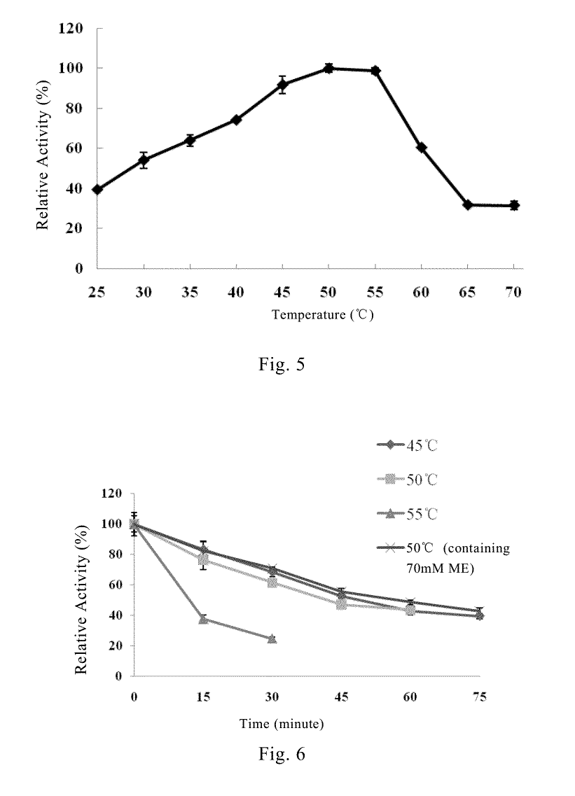

Enzymatic activity was detected according the above standard enzymatic activity detection steps at pH 7.0 and a temperature in the range of 25-80° C. Results were shown in FIG. 5. The optimum temperature of Xyl7 was in the range of 50-55° C., with an activity at 50□ slightly higher than that at 55□. Thus, the specific activity value of the enzyme at 50□ was used as a reference value and its relative activity was defined as 100%. The relative activity of the enzyme at each temperature was a ratio between the specific activity value of the enzyme at each temperature and the reference value. Xyl7 could retain 50% or more of the highest activity in the range of 30-60° C., indicating that it had a wide range of reaction temperature.

(7) Detection on Temperature Tolerance of Xyl7

The Xyl7 enzyme solution was stored in a buffer having an optimum pH 7.0 at different temperatures (55° C., 50° C., 45° C.) or at 50° C. with addition of 70 mM mercaptoethan for different times (15 min, 30 min, 45 min, 60 min, 75 min). Enzymatic activity was tested according to the above standard enzymatic activity detection steps (enzymatic activity was tested after reacting at pH7.0, 50° C. for 10 minutes). The enzyme solution which was not subjected to heat treatment was used as a control. Its specific activity tested under pH 7.0, 50° C. was used as a reference value and its relative activity was defined as 100%. The relative activity of the treatment group was a ratio between the relative activity value obtained after storing at different temperatures for different times (treatment conditions) and the reference value. Results were shown in FIG. 6. The activity of Xyl7 rapidly decreased to be less than 50% of the maximum activity after storing at 55° C. for 15 min, but slowly decreased when storing at 50° C. and 45° C. After storing at these temperatures for 45 min, the enzymatic activity decreased to be less than 50% of the maximum activity. When 70 mM mercaptoethan were added, the decrease rate of the enzymatic activity was further reduced.

(8) Effect of Different Chemical Agents and Metal Ions on the Enzymatic Activity of Xyl7

Various compounds were added into the reaction system to a final concentration of 10 mmol/L. Enzymatic activity was tested according to the above standard enzymatic activity detection steps. The specific activity value of the enzyme without treated by any chemical agent or metal ion was used as a reference value and its relative activity was defined as 100%. Effect of different chemical agents or metal ions on the activity of Xyl7 enzyme was exhibited by relative activity, which was a ratio between the specific activity value of the enzyme in an environment of the chemical agent or metal ion and the reference value. The results were shown in Table 4. K⁺, Mn²⁺, Cu²⁺ and Co²⁺ could activate the Xyl7, with K⁺ and Mn²⁺ producing an increase of about 20% for the activity of the enzyme. Ni²⁺, Zn²⁺, Fe²⁺ and EDTA obviously inhibited Xyl7, all of which could produce 70% or more of activity loss for the enzyme. Mg²⁺ showed no obvious action on Xyl7.

TABLE 4

| Metal Ion or Chemical Agent (10 mM) | Relative Activity (%) |
|---|---|
| PC | 100 ± 1.29 |
| K⁺ | 125.6 ± 11.98 |
| Mg²⁺ | 101.53 ± 3.02 |
| Ca²⁺ | 90.1 ± 12.82 |
| Fe²⁺ | 25.33 ± 0.46 |
| Cu²⁺ | 108.69 ± 2.99 |
| Zn²⁺ | 64.88 ± 1.36 |
| Co²⁺ | 109.14 ± 3.45 |
| Ni²⁺ | 44.25 ± 4.98 |
| Mn²⁺ | 125.73 ± 7.8 |
| Ba²⁺ | 90.71 ± 1.94 |
| Al³⁺ | 77.68 ± 2.32 |
| Fe³⁺ | 90.41 ± 3.36 |
| EDTA | 88.65 ± 1.62 |

(9) Hydrolysis of Xyl7 on Different Substrates

Various substrates with a final concentration of 2% (w/w) were treated by a suitable amount of enzymes at pH7.0 and 50□ for 10 minutes. Enzymatic activity was tested according to the above standard enzymatic activity detection steps. Results were shown in Table 5. Xyl7 showed a strong specificity to substrate and only exerted an obvious enzymatic activity on birch xylan and beech xylan. No detectable enzymatic activity was found for the other test substrates. This was consistent to the high substrate specificity of xylanase of the GH11 Family.

TABLE 5

| Substrate | Specific Activity (U/mg) |
|---|---|
| Beech xylan | 6340 ± 48 |
| Birch xylan | 4700 ± 57 |
| Microcrystalline cellulose powder | 0 |
| Carboxymethyl cellulose sodium | 0 |
| Laminarin | 0 |
| Barley dextran | 0 |
| Locust bean gum | 0 |

(10) TLC Analysis on Hydrolyzed Products of Birch Xylan by Xyl7

1% (w/w) Birch xylan was treated by 15 U Xyl7 at pH7.0 and 50□ for 10 min, 1 h, 4 h, and 12 h, respectively, to obtain the hydrolyzed products. Two products, each 5 μl, were subjected to TLC for identification. The standard samples were xylose, xylobiose, and xylotriose. The developing agent comprised ethyl acetate, acetic acid and water in a ratio of 2:1:1(V/V/V). The chromogenic agent was a solution of 1 mL aniline, 1 g diphenylamine and 5 mL 85% phosphoric acid dissolved in 50 mL acetone.

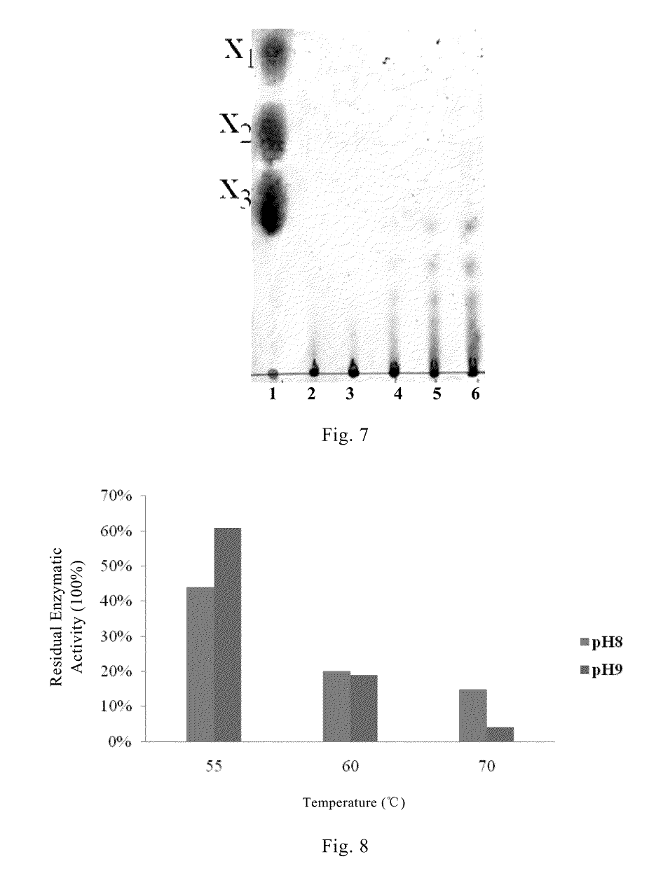

Results were shown in FIG. 7. When the action time was relatively short, the large polyxylan was preliminarily hydrolyzed by Xyl7 to oligoxylan and when the action time was relatively long, the polyxylan was hydrolyzed to xylo-oligosaccharide, mainly including xylobiose, xylotriose and xylotetraose. If the enzyme was excessive, the final hydrolyzed product was xylose. Xyl7 was demonstrated to act on the internal β-1,4-xylosidic linkage of the backbone of xylan in an endo-cleavage manner.

(11) Use of Xyl7 in Pulp Bleaching

For pulp bleaching, the xylanase is required to have no cellulase activity and to retain enzymatic activity at an intermediate or high temperature (such as 50-70° C. and in an alkaline environment (such as pH8-9) for one to two hours. Therefore, Xyl7 was treated at pH8 and pH9 and a temperature of 55° C., 60° C. and 70° C., respectively for two hours and its enzymatic activity was tested according to the above standard enzymatic activity detection steps (enzymatic activity was tested after reacting at pH7.0, 50□ for 10 minutes). The control was the un-treated enzyme solution. Its specific activity value was tested under pH7.0 and 50□ and used as a reference value and its relative activity was defined as 100%. The residual enzymatic activity was calculated as a ratio of the specific activity value of the enzyme obtained after various treatments and the reference value.

Results were shown in FIG. 8. It was found that Xyl7 could still retain a sufficiently high enzymatic activity for one to two hours even at a high temperature of from 50-70□ and in an alkaline environment of pH8-9. Thus, it could be used for pulp bleaching.

(12) Use of Xyl7 as a Feed Additive

As a feed additive, xylanase is required to retain its enzymatic activity in a pH environment of about 4 due to the acidic environment in the stomach of livestock, such as pig, and retain its enzymatic activity in a pH environment of about 6 due to the weak acidic environment in the digestive tract of chicken. Therefore, Xyl7 was tested for its residual enzymatic activity after treated in pH4 or pH5 at a 37° C. bath for 15, 30, 45 and 60 minutes. The residual enzymatic activity was a ratio between the specific activity of the enzyme obtained after various treatments and that of the un-treated enzyme.

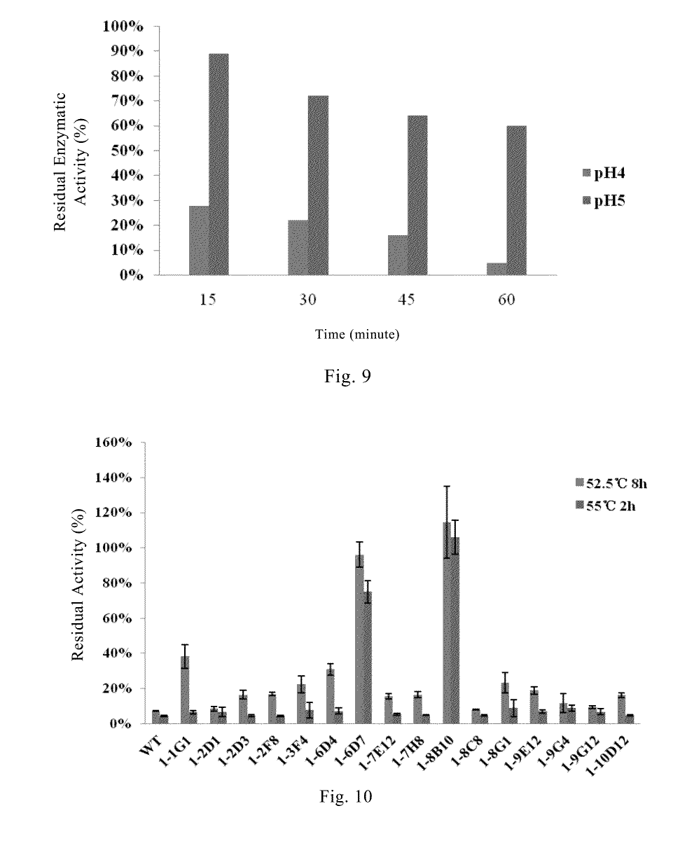

Results were shown in FIG. 9. It was found that Xyl7 could retain a sufficiently high enzymatic activity even in an acidic environment of pH 4-5. Thus, it could be used as a feed additive.

### Example 4

### Study on Property of Amino Acids 19-272 of Xyl7

A fragment of bases 58-801 (corresponding to amino acids 19-272 of the endo-1,4-β-xylanase) was amplified by the same method as described in Example 2 by using a forward primer (5′ TGAGACTCCATATGCAAGGTCCCACATGGACT 3′, SEQ ID NO: 10, with a Nde I recognition site CATATG added at its 5′ end) and a reverse primer (5′ GCGGAATTCTTATGGCGTAGGCGTGGTGCC 3′, SEQ ID NO: 11, with a EcoR I recognition site GAATTC added at its 5′ end). The fragment was cloned into a recombinant expression vector pET 28a and the vector was expressed in E. coli BL21(DE3). The expressed product, named Xyl7R3 hereinafter, was purified and its enzymatic activity was tested according to the same method as described in Example 3.

As shown in FIG. 14, the expression amount of amino acids 19-272 of Xyl7 (Xyl7R3) was obviously higher than that of Xyl7, and target protein of high purity could be obtained. Additionally, the specific activity of Xyl7R3 as detected by the standard enzymatic activity detection steps described in Example 3 was 8775 U/mg, demonstrating that it exhibited a comparable activity to the full length protein. The optimum temperature and tolerance on temperature of Xyl7R3 were almost identical to those of Xyl7.

### Example 5

### Directed Evolution of Xyl7

1. Construction of Xyl7 Random Mutation Library

A random mutation library was constructed by error-prone PCR. Error-prone PCR was conducted by using GeneMorph II Random Mutagenesis Kit and according to the methods provided in the kit. According to the method provided in the kit, 500 ng template DNA was added when performing the error-prone PCR and the mutation rate was adjusted to 1 mutation site per kb. The mutation library was constructed by performing two cycles of error-prone PCR with a library capacity of 40,000 for each cycle. The mutants obtained from each screening were re-screened and some resultant mutation sites were subjected to saturation mutation for each site.

2. Screen and Thermal Stability Test of Mutants of Xyl7 Having Improve Thermal Stability

Transformants were picked up by sterile toothpick to a LB liquid culture containing antibiotic and IPTG (in a final concentration of 1 mM) contained within the wells of a 96-well plate and cultured overnight at 37° C. and 200 rpm. Thallus was collected by centrifugation. 100 ul buffers having the optimum reaction pH were added to re-suspend the thallus. Clone from each well was divided into a control group and a treatment group, with 50 ul for each well. The control group was stored at 4° C. The treatment group was treated in a water bath having a suitable temperature for 2 hours. After adding 2% xylan to each well, the mixture was allowed to react under 37° C. for 1 hour. Enzymatic activity was detected according to the DNS method. After treatment, the treatment group of the wild type clone retained an enzymatic activity which was about 10˜20% of its control group. At this time the other clones could be screened to obtain the mutants having an improved thermal stability as compared to the wild type clone. The thermal stability of finally obtained mutants was tested according to the method described in Example 3. The overnight thallus was re-suspended and divided into two groups, which were the control and treatment groups, respectively. The control group was stored in 4° C. refrigerator and the treatment group was treated under specific temperatures for 2 hours. Then the enzymatic activity was detected. The residual enzymatic activity of the treatment group was calculated as a percentage of the enzymatic activity of the treatment group in relative to that of the control group.

After testing the thermal stability of the wild type sequence, it was found that, for the wild type sample, the enzymatic activity of the treatment group obtained after treating in a 52.5° C. water bath for two hours was 10˜20% of that of the control group, which complied with the requirement on screening. Thus, about 10,000 clones in the library were screened under this condition and 17 transformants were obtained, the treatment groups of which still retained 80% or more of the enzymatic activity of their respective control groups after heat treatment. To further verify the stability capacity of these positive transformants to temperature, two experiments for re-screening these clones were done. In the first experiment, the heat treatment temperature was kept at 52.5° C. but the treatment time was prolonged from 2 hours to 8 hours. In the second experiments, the heat treatment time was kept for 2 hours, but the treatment temperature was increased from 52.5° C. to 55° C.. The results after treatment were shown in FIG. 10. Two clones, numbered as 1-6D7 and 1-8B10, were further screened from the preliminarily screened 17 positive clones. Under the re-screening conditions, these two clones exhibited an obvious advantage on temperature stability as compared to the wild type and other positive clones. Mutant 1-6D7 contained a mutation K223E at amino acid 223, while mutant 1-8B10 contained mutations at three positions, which were K205E, K223T and A386S.

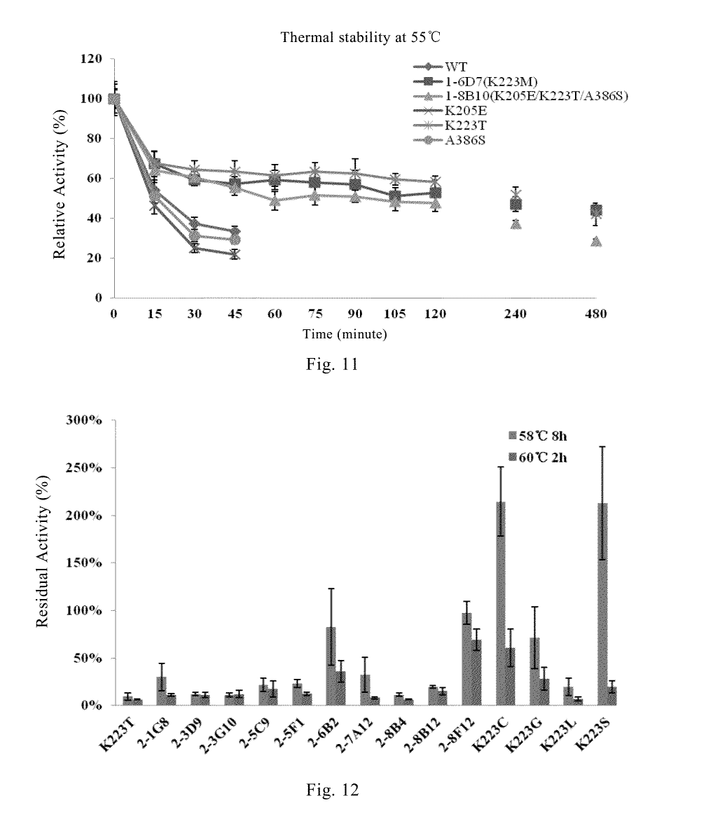

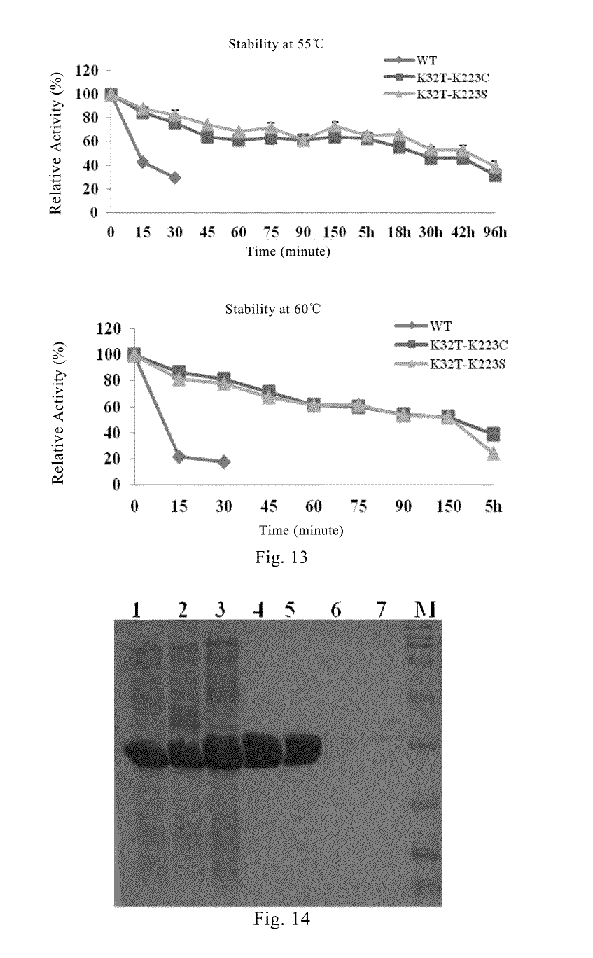

To study effect of each of these mutation sites on the temperature stability of the enzyme, totally 4 clones, each of which contained a single mutation for each site, were constructed, expressed and purified to obtain the protein. Each mutant was tested for its thermal stability. As shown in FIG. 11, mutants 1-6D7 (K223E) and 1-8D10 and the single-mutation K223T clone of the xylanase gene xyl7 exhibited an obvious improvement in thermal stability than the wild type and the other two single-mutation clones when kept at 55° C. The same trend was also observed when kept at 60° C. (results were not shown). It was found that the three mutants having an improved stability all contained a mutation at amino acid 223, with the mutant K223T having a most obvious improvement in stability, indicating that amino acid 223 was important to the enzymatic activity stability of the xylanase xly7. Saturation mutation was done at site 223. The single-mutation clone K223T was used a parent sequence to perform the second round of error-prone PCR to construct a random mutation library. Screening conditions were set up for the random mutation library for the xylanase gene xyl7 constructed from the second round of error-prone PCR, and mutants constructed from saturation mutation at site 223. More than 10,000 clones in the random mutation library were screened under 58° C. of heat treatment for 2 hours. About 20 mutants were obtained which exhibited obviously improved stability as compared to the starting strain (K223T). The enzymatic activity of the treatment group of these mutants was still 80% or more of that of the control group which was not undergone heat treatment. The saturation mutation clones at amino acid 223 of Xyl7 were screened and several mutants having an improved thermal stability as compared to the wild type K223T was obtained. Similar to the first round of re-screening, these mutants were re-screened under 58° C. for 8 hours or under 60° C. for 2 hours. Results showed that two mutants having an obviously improved thermal stability as compared to the starting strain xyl7-K223T were obtained from the random mutation library, which designated as 2-8F12 and 2-6B2. Mutants xyl7-K223C and xyl7-K223S were screened from the xyl7-223K saturation mutation library, which exhibited an obviously improved thermal stability than xyl7-K223T (FIG. 12). For the data described above, the residual relative activity of the mutant after high-temperature treatment was calculated by subtracting the relative activity of mutants without temperature treatment from that of the treatment group after high-temperature treatment. When screening mutants from the saturation mutation mutants, the starting strain used for constructing the mutant library was used as control. After the same treatment, mutants were considered to have an obviously improved enzymatic activity or thermal stability as compared to the wild type strain if its relative activity retained after treatment was obviously higher than the starting strain (wild type) undergone the same treatment.

After sequencing mutants 2-8F12 and 2-6B2, it was found that 2-8F12 contained a K32T mutation at amino acid 32 and 2-6B2 contained an E219D mutation at amino acid 219, in addition to K223T mutation in the parent. Mutant clones containing two or three mutations were constructed by combining the positive sites, K32T, E219D, K223C and K223S, of the resultant mutants. The clones were induced to express proteins and the proteins were purified. Thermal stability of the wild type xylanase xyl7 and each mutant protein at 55° C. and 60° C. were detected. Two mutants, xyl7-K32T/K223C and xyl7-K32T/K223S, which had an obviously improved stability than the wild type and other mutation clones, were obtained. It could be found from the data that the half life at 55° C. of the xylanase proteins from the two mutants were greatly increased from about 15 minutes of the wild type to 42 hours or above, which was totally increased about 250 folds. The half life at 60° C. was also increased from less than 10 minutes to 150 minutes or above.

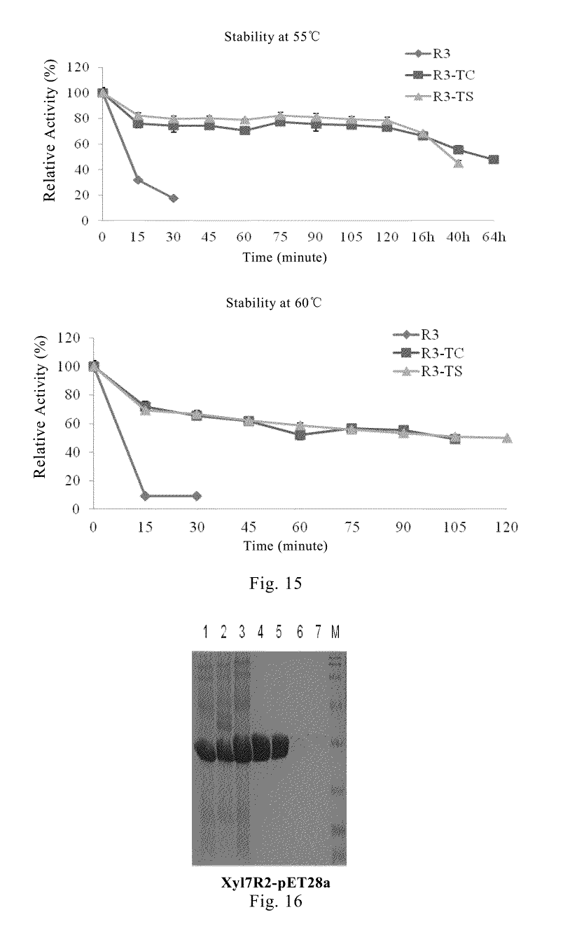

Additionally, a directed evolution library of wild type Xyl7 was constructed based on the amino acid sequence of Xyl7R3. From this library several site-directed mutations that could obviously enhance the stability of the enzyme were obtained, the mutants of which were named R3-TC(K32T/K223C) and R3-TS(K32T/K223S). As shown in FIG. 15, the half life at 55° C. of these two mutants were increased from about 10 minutes of the wild type to about 60 hours, which were increased about 360 folds. The half life at 60° C. was also increased from less than 10 minutes to 120 minutes.

Substitution mutations were made to the site of the fragment of amino acids 19-272 of Xyl7 corresponding to amino acid residue 37, 42, 80, 205, 219, 221, 222, 223, 228, 386 of SEQ ID NO:2, numbered according to amino acid position of SEQ ID NO:2. Mutations included one or more of N37D, S42N, M801, K205E, E219D, A221T, M22L, K223M or K223T, T228S and A386S. Results showed that these mutations retained the activity of the enzyme.

Substitution mutations were made to the site of the fragment of amino acids 19-272 of Xyl7 corresponding to amino acid residue 32 or 223 of SEQ ID NO:2, including single mutations K32T, K223E, K223C, K223S, and K223T, and double mutations K32T+K223C and K32T+K223S. Results showed that, similar to SEQ ID NO:2, the fragment of amino acid 19-272 of Xyl7 still showed an improved thermal stability after introducing the above mutations.

### Example 6

### Study on Property of the Truncated Amino Acid Fragments of Xyl7

A sequence shorter than amino acids 19-272 of SEQ ID NO:2 was designed based on the amino acid sequence of Xyl7 (SEQ ID NO:2) and expressed. The expressed sequence was amino acids 19-267 of SEQ ID NO:2, named Xyl7R2. According to item (3) “Detection of standard enzymatic activity” described in Example 3, the enzymatic activity of Xyl7R2 was 8560 U/mg. The results showed that R2 were active and had a high expression amount (FIG. 16).

All references mentioned in the present disclosure are incorporated by reference herein, as each of them is individually incorporated by reference. Additionally, it should be understood that various modifications or amendments could be made by the skilled in the art after reading the contents disclosed in the present disclosure. All these equivalences also fall within the scope defined in the attached claims of the subject application.

1. An isolated polypeptide selected from the group consisting of:
(a) a polypeptide the amino acid of which is set forth in SEQ ID NO:2;
(b) a polypeptide fragment consisting of amino acid residues 19-272 of SEQ ID NO:2;
(c) a polypeptide fragment consisting of amino acid residues 19-267 of SEQ ID NO:2;
(d) a polypeptide comprising amino acids 19-267 of SEQ ID NO:2;
(e) a polypeptide formed by substitution, deletion or addition of one or several amino acids in the amino acid sequence of (a), (b), (c) or (d) and having the function of the polypeptide of (a);
(f) a polypeptide formed by adding a tag sequence or a signal peptide sequence at the N or C terminus of the polypeptide of (a), (b), (c), (d) or (e); and
(g) a fusion protein containing the polypeptide of (a), (b), (c), (d) or (e).

2. The polypeptide of claim 1, wherein the polypeptide is selected from the group consisting of polypeptides containing amino acid substitution(s) at the site(s) corresponding to at least one of K32, N37, S42, M80, K205, E219, A221, M222, K223, T228 and A386 of SEQ ID NO:2.

3. The polypeptide of claim 1, wherein the polypeptide is selected from the group consisting of:
(1) the polypeptide in which a substitution mutation, K32T, is present at position corresponding to amino acid residue 32 of SEQ ID NO:2;
(2) the polypeptide in which a substitution mutation, N37D, is present at position corresponding to amino acid residue 37 of SEQ ID NO:2;
(3) the polypeptide in which a substitution mutation, S42N, is present at position corresponding to amino acid residue 42 of SEQ ID NO:2;
(4) the polypeptide in which a substitution mutation, M80I, is present at position corresponding to amino acid residue 80 of SEQ ID NO:2;
(5) the polypeptide in which a substitution mutation, K205E, is present at position corresponding to amino acid residue 205 of SEQ ID NO:2;
(6) the polypeptide in which a substitution mutation, E219D, is present at position corresponding to amino acid residue 219 of SEQ ID NO:2;
(7) the polypeptide in which a substitution mutation, A221T, is present at position corresponding to amino acid residue 221 of SEQ ID NO:2;
(8) the polypeptide in which a substitution mutation, M222L, is present at position corresponding to amino acid residue 222 of SEQ ID NO:2;
(9) the polypeptide in which a substitution mutation, K223M, is present at position corresponding to amino acid residue 223 of SEQ ID NO:2;
(10) the polypeptide in which a substitution mutation, K223T, is present at position corresponding to amino acid residue 223 of SEQ ID NO:2;
(11) the polypeptide in which a substitution mutation, K223C, is present at position corresponding to amino acid residue 223 of SEQ ID NO:2;
(12) the polypeptide in which a substitution mutation, K223S, is present at position corresponding to amino acid residue 223 of SEQ ID NO:2;
(13) the polypeptide in which a substitution mutation, K223G, is present at position corresponding to amino acid residue 223 of SEQ ID NO:2;
(14) the polypeptide in which a substitution mutation, K223L, is present at position corresponding to amino acid residue 223 of SEQ ID NO:2;
(15) the polypeptide in which a substitution mutation, T228S, is present at position corresponding to amino acid residue 228 of SEQ ID NO:2;
(16) the polypeptide in which a substitution mutation, A386S, is present at position corresponding to amino acid residue 386 of SEQ ID NO:2;
(17) the polypeptide in which substitution mutations, K205E, K223T and A386S, are present at positions corresponding to amino acid residues 205, 223 and 386 of SEQ ID NO:2;
(18) the polypeptide in which substitution mutations, K32T and K223T, are present at positions corresponding to amino acid residues 32 and 223 of SEQ ID NO:2;
(19) the polypeptide in which substitution mutations, K205E and K223T, are present at positions corresponding to amino acid residues 205 and 223 of SEQ ID NO:2;
(20) the polypeptide in which a substitution mutation, K223E, K223T, K223C, K223S, K223G or K223L, is present at position corresponding to amino acid residue 223 of SEQ ID NO:2;
(21) the polypeptide in which substitution mutations, K32T and K223C, are present at positions corresponding to amino acid residues 32 and 223 of SEQ ID NO:2; and
(22) the polypeptide in which substitution mutations, K32T and K223S, are present at positions corresponding to amino acid residues 32 and 223 of SEQ ID NO:2.

4. An isolated polynucleotide, which is selected from the group consisting of:
(1) the polynucleotide encoding the polypeptide of claim 1; and
(2) the polynucleotide complementary to the polynucleotide of (1).

5. A vector comprising the polynucleotide of claim 4.

6. A genetically engineering host cell comprising the vector of claim 5.

7. A method for producing a polypeptide selected from the group consisting of:
(a) a polypeptide the amino acid of which is set forth in SEQ ID NO:2;
(b) a polypeptide fragment consisting of amino acid residues 19-272 of SEQ ID NO:2;
(c) a polypeptide fragment consisting of amino acid residues 19-267 of SEQ ID NO:2;
(d) a polypeptide comprising amino acids 19-267 of SEQ ID NO:2;
(e) a polypeptide formed by substitution, deletion or addition of one or several amino acids in the amino acid sequence of (a), (b), (c) or (d) and having the function of the polypeptide of (a);
(f) a polypeptide formed by adding a tag sequence or a signal peptide sequence at the N or C terminus of the polypeptide of (a), (b), (c), (d) or (e);
(g) a fusion protein containing the polypeptide of (a), (b), (c), (d) or (e), comprising:
(a) culturing the host cell of claim 6 under the conditions suitable for the host cell to express the polypeptide; and
(b) isolating the polypeptide from the culture.

8. (canceled)

9. A method for degrading xylan, comprising mixing the polypeptide of claim 1 with xylan or a material containing xylan to allow the polypeptide to degrade the xylan or the xylan contained in a material containing xylan under suitable reaction conditions to oligoxylan or xylo-oligosaccharide or xylose.

10. The method of claim 9, wherein the xylan is selected from the group consisting of birch xylan and beech xylan.

11. The method of claim 9, wherein the material containing xylan is selected from the group consisting of pulp, feed and straw.

12. The method of claim 9, wherein the suitable reaction condition includes a pH of 3-12, preferably 5.5-10, more preferably about 7.0, and a temperature of 15-90° C., preferably 30-60° C., more preferably 50-55° C.

13. The method of claim 9, wherein the method further comprises adding an additive that regulating the enzymatic activity of the polypeptide to the mixture of the polypeptide and the xylan or the material containing xylan, wherein the additive is selected from the group consisting of K⁺, Mn²⁺, Cu²⁺ or Co²⁺, or substance that can be hydrolyzed to form K⁺, Mn²⁺, Cu²⁺ or Co²⁺ after adding to the substrate.

14. A composition comprising a safe and effective amount of the polypeptide of claim 1 and a bromatologically acceptable or industrially acceptable carrier.

15. A method for increasing the thermal stability of xylanase, comprising mutating the amino acid residue(s) of the xylanase polypeptide at position(s) corresponding to position 32 and/or 223 of SEQ ID NO:2, thereby obtaining a mutated xylanase having an improved thermal stability as compared the xylanase before mutation.

16. A method for screening a xylanase having an improved thermal stability, comprising:
(1) constructing a library comprising mutants of SEQ ID NO:2 or fragments of SEQ ID NO:2 comprising amino acids 19-267 based on SEQ ID NO:2 or fragments of SEQ ID NO:2 comprising amino acids 19-267; and
(2) testing the thermal stability of the mutants in the library;
wherein, after testing under the same test conditions, the mutant having a reduction degree of activity lower than that of the control by at least 5% is the mutant having an improved thermal stability.

17. The method according to claim 16, wherein the step of testing the thermal stability includes testing the enzymatic activity of the mutants and control at a pH of 3-12, preferably 5.5-10, more preferably about 7.0, and a temperature of 15-90° C., preferably 30-60° C., more preferably 50-55° C., and wherein the substrate for testing is selected from birch xylan and beech xylan.

18. The isolated polynucleotide of claim 4, wherein the polypeptide is selected from the group consisting of polypeptides containing amino acid substitution(s) at the site(s) corresponding to at least one of K32, N37, S42, M80, K205, E219, A221, M222, K223, T228 and A386 of SEQ ID NO:2; or
wherein the polypeptide is selected from the group consisting of:
(1) the polypeptide in which a substitution mutation, K32T, is present at position corresponding to amino acid residue 32 of SEQ ID NO:2;
(2) the polypeptide in which a substitution mutation, N37D, is present at position corresponding to amino acid residue 37 of SEQ ID NO:2;
(3) the polypeptide in which a substitution mutation, S42N, is present at position corresponding to amino acid residue 42 of SEQ ID NO:2;
(4) the polypeptide in which a substitution mutation, M801, is present at position corresponding to amino acid residue 80 of SEQ ID NO:2;
(5) the polypeptide in which a substitution mutation, K205E, is present at position corresponding to amino acid residue 205 of SEQ ID NO:2;
(6) the polypeptide in which a substitution mutation, E219D, is present at position corresponding to amino acid residue 219 of SEQ ID NO:2;
(7) the polypeptide in which a substitution mutation, A221T, is present at position corresponding to amino acid residue 221 of SEQ ID NO:2;
(8) the polypeptide in which a substitution mutation, M222L, is present at position corresponding to amino acid residue 222 of SEQ ID NO:2;
(9) the polypeptide in which a substitution mutation, K223M, is present at position corresponding to amino acid residue 223 of SEQ ID NO:2;
(10) the polypeptide in which a substitution mutation, K223T, is present at position corresponding to amino acid residue 223 of SEQ ID NO:2;
(11) the polypeptide in which a substitution mutation, K223C, is present at position corresponding to amino acid residue 223 of SEQ ID NO:2;
(12) the polypeptide in which a substitution mutation, K223S, is present at position corresponding to amino acid residue 223 of SEQ ID NO:2;
(13) the polypeptide in which a substitution mutation, K223G, is present at position corresponding to amino acid residue 223 of SEQ ID NO:2;
(14) the polypeptide in which a substitution mutation, K223L, is present at position corresponding to amino acid residue 223 of SEQ ID NO:2;
(15) the polypeptide in which a substitution mutation, T228S, is present at position corresponding to amino acid residue 228 of SEQ ID NO:2;
(16) the polypeptide in which a substitution mutation, A386S, is present at position corresponding to amino acid residue 386 of SEQ ID NO:2;
(17) the polypeptide in which substitution mutations, K205E, K223T and A386S, are present at positions corresponding to amino acid residues 205, 223 and 386 of SEQ ID NO:2;
(18) the polypeptide in which substitution mutations, K32T and K223T, are present at positions corresponding to amino acid residues 32 and 223 of SEQ ID NO:2;
(19) the polypeptide in which substitution mutations, K205E and K223T, are present at positions corresponding to amino acid residues 205 and 223 of SEQ ID NO:2;
(20) the polypeptide in which a substitution mutation, K223E, K223T, K223C, K223S, K223G or K223L, is present at position corresponding to amino acid residue 223 of SEQ ID NO:2;
(21) the polypeptide in which substitution mutations, K32T and K223C, are present at positions corresponding to amino acid residues 32 and 223 of SEQ ID NO:2; and
(22) the polypeptide in which substitution mutations, K32T and K223S, are present at positions corresponding to amino acid residues 32 and 223 of SEQ ID NO:2.

19. A vector comprising the polynucleotide of claim 18.

20. A genetically engineering host cell comprising an integrated polynucleotide including the polynucleotide of claim 4.

21. The method of claim 11, wherein the polypeptide is selected from the group consisting of polypeptides containing amino acid substitution(s) at the site(s) corresponding to at least one of K32, N37, S42, M80, K205, E219, A221, M222, K223, T228 and A386 of SEQ ID NO:2; or
wherein the polypeptide is selected from the group consisting of:
(1) the polypeptide in which a substitution mutation, K32T, is present at position corresponding to amino acid residue 32 of SEQ ID NO:2;
(2) the polypeptide in which a substitution mutation, N37D, is present at position corresponding to amino acid residue 37 of SEQ ID NO:2;
(3) the polypeptide in which a substitution mutation, S42N, is present at position corresponding to amino acid residue 42 of SEQ ID NO:2;
(4) the polypeptide in which a substitution mutation, M80I, is present at position corresponding to amino acid residue 80 of SEQ ID NO:2;
(5) the polypeptide in which a substitution mutation, K205E, is present at position corresponding to amino acid residue 205 of SEQ ID NO:2;
(6) the polypeptide in which a substitution mutation, E219D, is present at position corresponding to amino acid residue 219 of SEQ ID NO:2;
(7) the polypeptide in which a substitution mutation, A221T, is present at position corresponding to amino acid residue 221 of SEQ ID NO:2;
(8) the polypeptide in which a substitution mutation, M222L, is present at position corresponding to amino acid residue 222 of SEQ ID NO:2;
(9) the polypeptide in which a substitution mutation, K223M, is present at position corresponding to amino acid residue 223 of SEQ ID NO:2;
(10) the polypeptide in which a substitution mutation, K223T, is present at position corresponding to amino acid residue 223 of SEQ ID NO:2;
(11) the polypeptide in which a substitution mutation, K223C, is present at position corresponding to amino acid residue 223 of SEQ ID NO:2;
(12) the polypeptide in which a substitution mutation, K223S, is present at position corresponding to amino acid residue 223 of SEQ ID NO:2;
(13) the polypeptide in which a substitution mutation, K223G, is present at position corresponding to amino acid residue 223 of SEQ ID NO:2;
(14) the polypeptide in which a substitution mutation, K223L, is present at position corresponding to amino acid residue 223 of SEQ ID NO:2;
(15) the polypeptide in which a substitution mutation, T228S, is present at position corresponding to amino acid residue 228 of SEQ ID NO:2;
(16) the polypeptide in which a substitution mutation, A386S, is present at position corresponding to amino acid residue 386 of SEQ ID NO:2;
(17) the polypeptide in which substitution mutations, K205E, K223T and A386S, are present at positions corresponding to amino acid residues 205, 223 and 386 of SEQ ID NO:2;
(18) the polypeptide in which substitution mutations, K32T and K223T, are present at positions corresponding to amino acid residues 32 and 223 of SEQ ID NO:2;
(19) the polypeptide in which substitution mutations, K205E and K223T, are present at positions corresponding to amino acid residues 205 and 223 of SEQ ID NO:2;
(20) the polypeptide in which a substitution mutation, K223E, K223T, K223C, K223S, K223G or K223L, is present at position corresponding to amino acid residue 223 of SEQ ID NO:2;
(21) the polypeptide in which substitution mutations, K32T and K223C, are present at positions corresponding to amino acid residues 32 and 223 of SEQ ID NO:2; and
(22) the polypeptide in which substitution mutations, K32T and K223S, are present at positions corresponding to amino acid residues 32 and 223 of SEQ ID NO:2.


SEQUENCE LISTING (29 sequences)

SEQ ID NO: 1 - DNA, 1518 bp, Unknown
  Intestinal tract metagenome of termite
atgaaaaaactcattgctctttcattcatagcggcatttttcgcgccgctattcgctcaaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 2 - protein, 505 aa, Unknown
  Intestinal tract metagenome of termite
MKKLIALSFIAAFFAPLFAQGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 3 - DNA, 31 bp, Artificial sequence
  Primer
gagactccatatgcaaggtcccacatggact

SEQ ID NO: 4 - DNA, 26 bp, Artificial sequence
  Primer
cggaattcttacctcaccataaccct

SEQ ID NO: 5 - protein, 5 aa, Artificial sequence
  Tag sequence
RRRRR

SEQ ID NO: 6 - protein, 6 aa, Artificial sequence
  Tag sequence
HHHHHH

SEQ ID NO: 7 - protein, 8 aa, Artificial sequence
  Tag sequence
DYKDDDDK

SEQ ID NO: 8 - protein, 8 aa, Artificial sequence
  Tag sequence
WSHPQFEK

SEQ ID NO: 9 - protein, 10 aa, Artificial sequence
  Tag sequence
WQKLISEEDL

SEQ ID NO: 10 - DNA, 32 bp, Artificial sequence
  Primer
tgagactccatatgcaaggtcccacatggact

SEQ ID NO: 11 - DNA, 30 bp, Artificial sequence
  Primer
gcggaattcttatggcgtaggcgtggtgcc

SEQ ID NO: 12 - DNA, 1461 bp, Artificial sequence
  Synthesized coding sequence of N37D mutant of Xy17
caaggtcccacatggactactagcacaatacaagcttacaacggctacgactacgagctttggaaccaaaacaacgcaggcaccgttagcatgaaactcacgggagatgatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 13 - protein, 486 aa, Artificial sequence
  Synthesized amino acid sequence of N37D mutant of Xy17
QGPTWTTSTIQAYNGYDYELWNQNNAGTVSMKLTGDDGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 14 - DNA, 1461 bp, Artificial sequence
  Synthesized coding sequence of S42N mutant of Xy17
caaggtcccacatggactactagcacaatacaagcttacaacggctacgactacgagctttggaaccaaaacaacgcaggcaccgttagcatgaaactcacgggagataatggatcaggtgccaacgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 15 - protein, 486 aa, Artificial sequence
  Synthesized amino acid sequence of S42N mutant of Xy17
QGPTWTTSTIQAYNGYDYELWNQNNAGTVSMKLTGDNGSGANAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 16 - DNA, 1461 bp, Artificial sequence
  Synthesized coding sequence of M80I mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatataagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 17 - protein, 486 aa, Artificial sequence
  Synthesized amino acid sequence of M80I mutant of Xy17
QGPTWTTSTIQAYNGYDYELWNQNNAGTVSMKLTGDNGSGASAVGGTFTATWSNTQNVLFRSGKKWGSSSNQNHTQIGNISINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 18 - DNA, 1461 bp, Artificial sequence
  Synthesized coding sequence of E219D mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgatgtggcgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 19 - protein, 486 aa, Artificial sequence
  Synthesized amino acid sequence of E219D mutant of Xy17
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYDVAMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 20 - DNA, 1461 bp, Artificial sequence
  Synthesized coding sequence of A221T mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtgacgatgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 21 - protein, 486 aa, Artificial sequence
  Synthesized amino acid sequence of A221T mutant of Xy17
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVTMKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 22 - DNA, 1461 bp, Artificial sequence
  Synthesized coding sequence of M222L mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagtagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgttgaaggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 23 - protein, 486 aa, Artificial sequence
  Synthesized amino acid sequence of M222L of Xy17
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVALKVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 24 - DNA, 1461 bp, Artificial sequence
  Synthesized coding sequence of K223M mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgatggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 25 - protein, 486 aa, Artificial sequence
  Synthesized amino acid sequence of K223M mutant of Xy17
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMMVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 26 - DNA, 1461 bp, Artificial sequence
  Synthesized coding sequence of T228S mutant of Xy17
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcagaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaataaatggcatgaacttggcatgaagatggacggtccgttatatgaggtggcgatgaaggttgaatcttattccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtagcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 27 - protein, 486 aa, Artificial sequence
  Synthesized amino acid sequence of T228S mutant of Xy17
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNKWHELGMKMDGPLYEVAMKVESYSGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVAASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR

SEQ ID NO: 28 - DNA, 1461 bp, Artificial sequence
  Synthesized coding sequence of K205E,K223T, A386S mutant of Xyl7
caaggtcccacatggactactagcacaatacaagcttacaacggctacgactacgagctttggaaccaaaacaacgcaggcaccgttagcatgaaactcacgggagataatggatcaggtgccagcgcggtaggcgggacgtttacggcaacatggagcaacacgcaaaacgtgcttttccgctccggtaaaaaatggggatccagcagtaaccaaaaccatacgcaaatcggcaatatgagcattaactttgccgctacatggtcttccaccgacaacgtgaaaatgctcggcgtttacggctgggcgtatttcacatcggcaaatgtgccgacaaaacaagaaaacggtacaaacgctagtttctccaaccaaatcgaatactatattattcaagataggggtagttataatccggcttcgggaggcactaacgccaaaaaatacggttcggctaccattgacggcattgcgtatgatttctatgtatgcgacagaatcaatcagcctatgttaaccggcaacgggaatttcaaacaattcttcagcgttcctcaaagcgcttctgcccacagaacaagcggtacgatttctgtttcacagcactttaatgaatggcatgaacttggcatgaagatggacggtccgttatatgaggtggcgatgacggttgaatcttataccggcagtgggagtagcaacggttcggcaacagttacgaagaatcttttgaccattggaggcaccacgcctacgccaagttccaactcaggcggcacaacaagcagctctagcagagcatcttcttcaagcagagcatcttcttcaagcaatgcggcggttcaagcaactacttgcaaaacgcctttgataacatacccaacaagcacggtgccttcagatccctacaccgcctgctttaaatatacagatgacaaatgctatgtttgcaaagtggaaaatgaaggcgaatttgaaggcaacatgaacacttgcggctctggctgggtgtgggacggcacgcagatagacaacaatttaagggatggctattggtatcaagaagtgccttgccctgcaggttcatcttctagccgttcaagcagctcttctgtagcggtttcatcctctagccgttcaagcagctcttccgtatcggcatcttcttctagccgttcaagcagttcgtctgcatcggttagcagttcatcggaagagactcctatcagggtgctccacacaattccttcaaaattccgcgttcgctcgctgaacaacggagctttgcagattgaaagcaatgcggatgttgtgctttatttatacgataccaaaggaaaaatggctcaaaaaattgaagccccaacgggttcaagcattgtaaagctatctgttcccgctggaatctatatagtgagaaacgttcaaaccaaagaaaaactaagggttatggtgaggtaa

SEQ ID NO: 29 - protein, 486 aa, Artificial sequence
  Synthesized amino acid sequence of K205E,K223T, A386S mutant of Xyl7
QGPTWTTSTIQAYNGYDYELWNQNNAGTVSMKLTGDNGSGASAVGGTFTATWSNTQNVLFRSGKKWGSSSNQNHTQIGNMSINFAATWSSTDNVKMLGVYGWAYFTSANVPTKQENGTNASFSNQIEYYIIQDRGSYNPASGGTNAKKYGSATIDGIAYDFYVCDRINQPMLTGNGNFKQFFSVPQSASAHRTSGTISVSQHFNEWHELGMKMDGPLYEVAMTVESYTGSGSSNGSATVTKNLLTIGGTTPTPSSNSGGTTSSSSRASSSSRASSSSNAAVQATTCKTPLITYPTSTVPSDPYTACFKYTDDKCYVCKVENEGEFEGNMNTCGSGWVWDGTQIDNNLRDGYWYQEVPCPAGSSSSRSSSSSVAVSSSSRSSSSSVSASSSSRSSSSSASVSSSSEETPIRVLHTIPSKFRVRSLNNGALQIESNADVVLYLYDTKGKMAQKIEAPTGSSIVKLSVPAGIYIVRNVQTKEKLRVMVR